(19) United States
(12) Patent Application Publication
(10) Pub. No.: US 2003/0027870 A1
(43) Pub. Date: Feb. 6, 2003

(54) Fullerene derivatives that modulate nitric oxide synthase and clamodulin activity

(76) Inventors: Stephen R. Wilson, Chatham, NJ (US); Donald J. Wolff, Bridgewater, NJ (US); David I. Schuster, Wilton, CT (US); Christine F. Richardson, Hastings-on-Hudson, NY (US); Alexandru Dragos Petru Papoiu, Piscataway, NJ (US); John Michael Alford, Lakewood, CO (US)

Correspondence Address: GREENLEE WINNER AND SULLIVAN P C, 5370 MANHATTAN CIRCLE, SUITE 201, BOULDER, CO, 80303, US

(21) Appl. No.: 10/147,453

(22) Filed: May 15, 2002

Related U.S. Application Data
(60) Provisional application No. 60/291,462, filed on May 15, 2001.

(51) Int. Cl. A61K 31/135; A61K 31/05

(52) U.S. Cl. 514/656; 514/732

(57) ABSTRACT

This invention provides novel fullerene derivatives, particularly fullerene derivatives that are water-soluble, that modulate the activity of nitric oxide synthase (NOS) and/or calmodulin. The invention provides methods for modulating NOS activity and particularly provides methods for inhibiting NOS activity, by contacting one or more fullerene derivatives of this invention with cells or tissue that exhibit NOS activity. In a specific embodiment, the invention provides water-soluble fullerene derivatives that are selective inhibitors of neuronal NOS or iNOS. Preferred water soluble fullerenes have substituents that contain one or more amine groups, amine cationic groups. More generally, water-soluble fullerenes of this invention contain one or more polar, one or more charged or more zwitterionic groups

### CROSS-REFERENCE TO RELATED APPLICATIONS

This application takes priority under 35 U.S.C. 119(e) from U.S. provisional application 60/291,462, filed May 15, 2001 which is incorporated by reference in its entirety herein.

### STATEMENT REGARDING FEDERALLY SPONSORED RESEARCH

This application was made at least in part through funding from the National Institutes of Health grants ES06897 and HL 54768 and the National Science Foundation grant CHE 9712735. The United States government has certain rights in this invention.

### BACKGROUND OF THE INVENTION

Fullerenes (Kroto et al. (1985) Nature 318:162) are a new class of hollow, closed shell, all carbon molecules. These materials can now be prepared in quantities sufficient to examine their biological activity (Kratschner, W. et al. (1990) Nature 347:354-358).

Fullerenes contain a large number of conjugated double bonds that are highly reactive with free radicals and reactive oxygen species. As a consequence of this reactivity, C₆₀ has been described as a “radical sponge” (Krusic et al. (1991) Science 1:1183). Compounds exhibiting antioxidant and radical scavenging properties have widely-recognized, significant medical and therapeutic applications. Biological applications of fullerenes, in general, and as antioxidants and free radical scavengers, in particular, have been limited by the extreme hydrophobicity of the fullerenes which are soluble in aromatic solvents, but not in water or aqueous media typical of biological systems. To be useful in biological applications, fullerenes must be derivatized with appropriate hydrophillic groups to render the derivatized fullerenes at least partially soluble in aqueous media. Water-soluble fullerene derivatives have been made by derivatizing the fullerene sphere with hydroxyl or carboxylic acid groups.

Certain water-soluble fullerene derivatives have been reported to have specific biological activities. Polyhydroxylated and hemiketal C₆₀ derivatives (C₆₀ (OH)ₙ, where n is 12 and C₆₀ (OH)ₙOₘ where n is 18-20 and m is 3-7, respectively) were reported to exhibit antioxidant properties functioning as OH radical scavengers. (Dugan et al. (1996) Neurobiology of Disease 3:129-135). These fullerene derivatives were reported to provide neuroprotection against excitotoxic injury in cortical neuronal cultures and to reduce serum deprivation-induced apoptosis of neuronal cells. A later report by the same authors indicates that the results observed with the polyhydroxylated and hemiketal C₆₀ derivatives exhibited “considerable synthesis lot-to-lot variability” in solubility and biological effect. (Dugan et al. (1997) Proc. Natl. Acad. Sci. 94:9434-9439.) The authors suggest that the variability observed is due to uncontrolled differences in the number and location of hydroxy and hemiketal moieties.

Additional reports indicate that polyhydroxylated fullerenes function as antioxidants to block hydrogen peroxide-induced inhibition of population spikes in hippocampus (Tsai (1997) J. Pharm. Pharmacol. 49:438-445)and to attenuate exsanguination-induced bronchoconstriction via an increase in free radicals (Lai and Chang (1997) J. Auton. Pharmacol. 17:229-235). Lin et al. (1999) J. Neurochem. 72(4):1634-1640 report that a water-soluble carboxyfullerene (characterized as the c-regioisomer) prevented iron-induced oxidative injury in the nigrostriatal dopaminergic system of anesthetized rats. The authors suggest that this potent antioxidant activity of carboxyfullerene may be therapeutically useful in the treatment of Parkinson's disease.

Dugan et al. 1997 supra reports that two fullerene derivative regioisomers with either C₃ or D₂ symmetry having three malonic acid groups per molecule are efficient free radical scavengers in solution (aqueous) and inhibit neuronal death in cell cultures caused by exposure to certain cytotoxic agents (NMDA or AMPA) or by oxygen-glucose deprivation. The C₃ isomer was reported to be more effective than the D₃ isomer for inhibition of cell death in culture. In addition the C₃ isomer was reported to inhibit apoptotic cell death induced by serum deprivation or by exposure to the Alzheimer's disease amyloid protein Aβ₄₂. The C₃ regioisomer was further reported to delay death and functional deterioration in transgenic mice carrying the human superoxide dismutase mutant that is responsible for a form of familial amyotropic lateral sclerosis.

Satoh et al. (1997) Eur. J. Pharmacol. 327:175-181 report that the dimalonic acid derivative of C₆₀ causes specific inhibition of acetylcholine- and substance P-induced relaxation in endothelium-containing thoracic aorta and of S-nitroso-N-acetyl-penicillamine-induced relaxation in endothelium-denuded thoracic aorta of rabbits. The authors state that this fullerene derivative generates superoxide that reacts with and destroys nitric oxide and as a result inhibits muscle relaxation mediated by nitric oxide.

Water-soluble fullerene derivative have also been reported to have biological activity as inhibitors of cysteine proteinases and serum proteinases (Ruoff, R. S. et al. (1993) J. Phys. Chem. 97:3379-3386; Tokuyama, H. et al. (1993) J. Am. Chem. Soc. 115:7918-7923), and to exhibit HIV antiviral activity (Friedman, S. H. et al. (1993) J. Am. Chem. Soc. 115:6506-6509; Schinazi, R. F. et al. (1993) Antimicrob. Agents Chemother. 37:1707-1710; Wilson, S. R. (2000) “Biological Aspects of Fullerenes,” in Fullerenes: Chemistry, Physics and Technology, Kadish, K. M. and Ruoff, R. S., Eds., John Wiley and Sons, New York, pp. 431-436).

The present invention relates to fullerene derivatives particularly to water-soluble fullerene derivatives which regulate nitric oxide synthase (NOS), in particular to fullerene derivatives that inhibit NOS isoforms and more particularly to fullerene derivatives that exhibit selective inhibition of NOS isoforms. The invention also relates to fullerene derivatives that inhibit calmodulin activity and act as calmodulin antagonists.

Nitric oxide (NO) is an important effector molecule in the nervous, immune, and cardiovascular systems (Nathan, C. (1992) FASEB J. 6:3051-3064). Compounds that modulate the level of nitric oxide generated are attractive targets in pharmaceutical research.

Nitric oxide is synthesized by nitric oxide synthase (NOS), a cytochrome P450-like heme protein that utilizes tetrahydrobiopterin, FAD, and FMN as cofactors to catalyze the NADPH-dependent oxidation of L-arginine to form citrulline and NO (Griffith, O. W. and Stuehr, D. J. (1995) Annu. Rev. Physiol. 57:707-736; Bryk, R. and Wolff, D. J. (1999) Pharmacol. Ther. 84:157-178). Nitric oxide synthase exists in three major isozymic forms: neuronal NOS (nNOS) isolated from neurons and GH3 pituitary cells which is constitutive and Ca²⁺- and calmodulin-dependent; cytokine-inducible NOS (iNOS) widely distributed and characterized extensively from macrophage; and endothelia NOS (eNOS) isolated from endothelia which is constitutive and Ca²⁺- and calmodulin-dependent (Forstermann, U. et al. (1995) Biochem. Pharmacol. 9:1321-1332; Wolff, D. J. and Datto, G. (1992) Biochem. J. 284:201-206; Stuehr, D. J. et al. (1991) Proc. Natl. Acad. Sci. USA 88:7773-7777; Hevel, J. M. et al. (1991) J. Biol. Chem. 266:22789-22791; Lamas, S. et al. (1992) Proc. Natl. Acad. Sci. USA 87:3629-3632).

In particular circumstances excess NO generated by nNOS and/or iNOS can be cytotoxic. Overproduction of NO by nNOS has been implicated in the tissue damage accompanying ischemia-reperfusion following stroke and in diverse neurodegenerative disorders (Huang, Z. et al. (1990) Science 265:1883-1888; Hantraye, P. et al. (1996) Nat. Med. 2:1017-1021). Overproduction of NO by iNOS has been implicated in septic shock, in diverse autoimmune disorders (e.g., multiple sclerosis) and in arthritis (Kilbourn, R. G. et al. (1990) Proc. Natl. Acad. Sci. USA 87:3629-3632; Hooper, D. C. et al. (1997) Proc. Natl. Acad. Sci. USA 94:2528-2533; McCartney-Francis, N. et al. (1993) J. Exp. Med. 178:749-754). In contrast, function of eNOS is necessary for the maintenance of appropriate tissue blood flow and inhibition of eNOS will elicit sever hypertension. Attention has thus been directed to the development of isoform selective NOS inhibitors that will inhibit nNOS and/or iNOS to a greater extent than eNOS.

There is a need in the art for pharmaceutically acceptable compounds that modulate the activity of NOS for the treatment of disorders and pathological conditions that are associated with nitric oxide levels. In particular, there is a significant need in the art for NOS inhibitors to treat conditions and pathological conditions associated with the overproduction of nitric oxide. Of significant interest are selective NOS inhibitors which control undesirable levels of NO, particularly in certain tissue, and the disorders and pathologies associated with them, yet which exhibit minimal inhibition of beneficial NOS, particularly eNOS, activity.

Calmodulin is a Ca²⁺-binding protein that is involved in the regulation of various activities in biological systems. Calmodulin is involved, for example, in the regulation of the activity of various enzymes, including, as noted above, certain isoforms of NOS, and as such can influence various biological activities. Calmodulin antagonist, which inhibit calmodulin activity, have been suggested in the art for various therapeutic applications. Certain calmodulin antagonists are reported to functions as sedatives or tranquillizers (U.S. Pat. No. 2,645,610). Calmodulin antagonists have also been reported to inhibit cell proliferation (Hidako et al. (19) Proc. Natl. Acad. Sci. 78:4354-4357) and to stimulate immune response (U.S. Pat. No. 5,698,518). In addition, certain calmodulin antagonists have been reported to function in the treatment of dermatitis (U.S. Pat. No. 5,583,143). There is a continuing need in the art for pharmaceutically acceptable compounds which inhibit calmodulin activity and which as a result can be employed to ameliorate a disease or condition which is associated with calmodulin activity.

### SUMMARY OF THE INVENTION

This invention provides novel fullerene derivatives, particularly fullerene derivatives that are water-soluble, that modulate the activity of nitric oxide synthase (NOS). In one aspect of the invention, fullerene derivatives of this invention inhibit the activity of NOS. In a specific embodiment, fullerene derivatives useful for NOS modulation carry one or two or more positively charged functional groups that may be employed in the form of cations and pharmaceutically acceptable salts of such cations or one or more zwitterionic groups. In a second aspect, fullerene derivatives of this invention exhibit bimodal function to modulate NOS activity and to function as neuroprotective agents. The invention thus provides methods for modulating NOS activity and particularly provides methods for inhibiting NOS activity, by contacting one or more fullerene derivatives of this invention with cells or tissue that exhibit NOS activity.

In another aspect the water-soluble fullerenes of this invention, affect the activity of calmodulin and specifically inhibit the activity of calmodulin. In a specific embodiment, fullerene derivatives useful for inhibition of calmodulin carry one or more positively charged functional groups that may be employed in the form of cations and pharmaceutically acceptable salts of such cations or one or more zwitterionic groups.

Fullerene derivatives of this invention are illustrated schematically by Formula I:

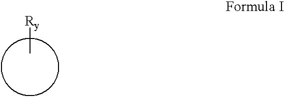

where the fullerene group is represented schematically as a circle, and substituents on the fullerene are schematically represented as R groups without indication of specific locations or modes of binding of the R groups to the fullerene. The variable R, independent of other R groups, represents a functional group and y represents the number of functional groups on the fullerene. In general the fullerenes of Formula I can be any size, but preferably the fullerene is C₆₀ or C₇₀ and more preferably the fullerene is C₆₀. At least one of the R groups present on the fullerene of Formula I is a water-solubilizing group that can be located at any position on the fullerene that can be derivatized. An R group can contain one or more functional groups that facilitate solubilization of the derivatized fullerene in water or aqueous solutions. Water-solubilizing functional groups most generally are or comprise hydrophillic groups. Hydrophillic groups include functional moieties that are polar, are positively or negatively charge or are zwitterionic (containing both an anionic and a cationic moiety, but overall charge neutral).

More preferably, two or more R groups on the fullerene are water-solubilizing groups. Most preferably, three or more R groups on the fullerene are water-solubilizing groups. Preferred water-solubilizing groups are positively charged or zwitterionic. All of the R groups of the fullerene may be the same or they may be different. In preferred embodiments, all of the R groups are the same and all are water-solubilizing. In specific preferred embodiments, R groups are positively charged groups R³⁰ typically associated in salts with one more anions (A⁻) to neutralize the positive charge, negatively charged groups R⁻, typically associated in salts with one or more cations C⁺ to neutralize the negative charge or zwitterionic, shown schematically as B⁻—R—D⁺, with separated charged moieties. Examples of zwitterions are amino acid groups —CH(O)(NH₃⁺)(COO⁻) where Q is a given side chain. Positively charged R groups can, for example, carry one or more positively charged amine groups:

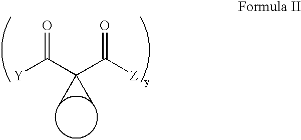

where R′ is H, an alkyl or aryl group. Negatively charged R groups can carry, for example, one or more carboxylate moieties (—COO⁻), one or more —SO₃²⁻, or one or more PO₄⁻, —P(OH)O₃⁻ or ⁻P(OH)₂O₂⁻ moieties. Zwitterionic R groups contain one positively charged moiety (e.g., a charged amino group) and one negatively charged moiety (e.g., —COO⁻, SO₃⁻) or they may contain more than one pair of each of a positively charged moiety and a negatively charged moiety.

In specific prefer red embodiments, fullerenes are derivatized with malonates or derivatives thereof, as illustrated in Formula II:

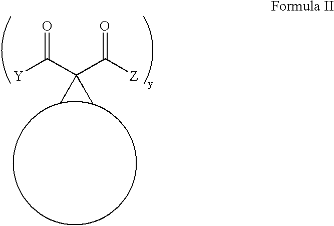

where y is the number of malonate functions on the fullerene and Y and Z, independently of one another and other Y or Z substituents, are selected among others, from the groups consisting of —OH, —O⁻, —N(R′)₂—O—(CH₂)ₙ—N(R′)₂, alkylanines, cationic amines, —O—(CH₂)ₙ—N(R′)₃⁺, amides, alkyl groups, aryl groups, alkoxy groups, aryloxy groups, thioalkyl groups (—S-alkyl), —O-aryl groups, or —S-aryl groups, ether groups, polyether groups, polyols, and various water-solubilizinig polymers. Y and Z may be the same or different groups. Alkyl groups and aryl groups can be optionally substituted with one or more halogens, —NO₂ groups, —N(R′)₂ groups, —N(R′)₃⁺ groups, —COOH groups, —COO⁻ groups, or —SO₃⁻ groups, where n is an integer from 1 to 20 and R′, independently of other R′ is —H, -alkyl or -aryl which in turn can be optionally substituted. The malonate functions can be distributed over the surface of the fullerene to provide various regioisomers. In preferred embodiments of the fullerene derivatives of Formula II, y is 1, 2, 3, 4, 5, or 6. In further preferred embodiments, Y and Z are selected from —O—(CH₂)ₙ—N(R′)₂, —O—(CH₂)ₙ—N(R′)₃⁺, —O-alkyl, —O-aryl which is optionally substituted with one or more halogens, —NO₂ groups, —N(R′)₂ groups, —N(R′)₃⁺ groups, —COOH groups, —COO⁻ groups, or —SO₃⁻ groups, where n is an integer from 1 to 20 preferably an integer from 1-10 and more preferably an integer from 1-6) and R′, independently of other R′, is —H, -alkyl or -aryl which in turn can be optionally substituted. In additional preferred embodiments at least one of R contains at least one zwitterionic group, such an amino acid group. In specific embodiments y is 1 or y is 3. In yet other specific embodiments, y is 1 or 3 and Y and Z are selected from —O—(CH₂)ₙ—N(R′)₂, —O—(CH₂)ₙ—N(R′)₃⁺, —O-alkyl, where n is 1-20, preferably 1-10 and more preferably 1-6 and the alkyl group is a lower alkyl groups having from 1 to 6 carbon atoms. In more specific embodiments, y is 1 or 3 and Y and Z are selected from —O—(CH₂)₃—NH₂, —O—(CH₂)₃—NH₃⁺, or —O—CH₂—CH₃. This invention specifically provides novel water-soluble fullerene derivatives of the formulas herein.

Fullerenes of this invention which inhibit NOS preferably have IC₅₀ values (the concentration of fullerene derivative which inhibits NOS activity by 50%) lower than about 500 μM, preferably lower than 100 μM, more preferably lower than about 10 μM and yet more preferably lower than about 2 μM. In a particular embodiment, fullerene derivatives of this invention exhibit selective modulation, e.g., selective inhibition, for the different isoforms of NOS (nNOS, iNOS or eNOS). Selective nNOS or iNOS inhibitors, for example, exhibit IC₅₀ binding or inhibitory parameters that are lower than the corresponding values for eNOS. Preferably, fullerene derivatives that are selective inhibitors of nNOS or iNOS exhibit at least 2-fold higher inhibition (indicated by 2-fold lower IC₅₀ values) for nNOS and/or iNOS than for eNOS. More preferred selective inhibitors of nNOS or iNOS exhibit at least 10-fold higher inhibition for nNOS and/or iNOS than for eNOS. Yet more preferred selective inhibitors of nNOS or iNOS exhibit at least 50-fold higher inhibition for nNOS and/or iNOS than for eNOS. Most preferred selection inhibitors of nNOS or iNOS exhibit at least 100-fold higher inhibition for nNOS and/or iNOS than for eNOS.

The invention also provides pharmaceutical compositions comprising a pharmaceutically acceptable carrier and one or more of the water-soluble fullerene derivatives of this invention which inhibit NOS present in the composition in an amount or a combined amount effective for inhibiting NOS. These pharmaceutical compositions are useful in the treatment of various disorders and pathological conditions characterized by overproduction of nitric oxide, including but not limited to, septic shock, autoimmune disorders, arthritis, reperfusion injury (e.g., reperfusion injury following stroke), and neurodegenerative disorders, such amyotrophic lateral sclerosis. The pharmaceutical compositions which comprise selective inhibitors of nNOS are particularly useful for treatment of neuronal disorders.

Fullerene derivatives of this invention which inhibit calmodulin (e.g., are calmodulin antagonists) preferably exhibit IC₅₀ values of 250 nM or less, and more preferably exhibit values of 100 nM or less. The invention also provides pharmaceutical compositions comprising a pharmaceutically acceptable carrier and one or more of the water-soluble fullerene derivatives of this invention which inhibit calmodulin present in the composition in an amount or a combined amount effective for inhibiting calmodulin activity. These pharmaceutical compositions are useful in the treatment of various disorders and pathological conditions associated with the activity of calmodulin including, but not limited to, treatment of dermatitis, stimulation of the immune system, inhibiting or retarding cell proliferation. Pharmaceutical compositions of this invention containing one or more fullerene derivatives that are calmodulin inhibitors can be employed as tranquillizers and/or sedatives.

In a further specific embodiment, the invention provides fullerene derivatives that exhibit bimodal activity, i.e., a single fullerene derivative modulates NOS activity as well as scavenging reactive oxygen species (ROS). Preferably, fullerene derivatives of this embodiment exhibit antioxidant activity greater than trolox as assayed in superoxide dismutase assays. Preferably, fullerene derivatives of this embodiment inhibit NOS activity and function as antioxidants. Pharmaceutical compositions comprising therapeutically effective amounts of fullerene derivative exhibiting bimodal activity are additionally useful in the treatment of various disorders and pathological conditions which are associated with or caused by the presence of reactive oxygen species, including, but not limited to Parkinsons's disease, ischemia and reperfusion injury, Alzheimer's disease, glaucoma, inflammatory conditions, and cardiac and circulatory disorders. Additionally, pharmaceutical compositions comprising fullerene derivatives of this invention exhibiting NOS modulation, alone or in combination with antioxidant function, are useful for the treatment and/or prevention of cancers and as anti-aging compositions.

The invention also provides methods for treating diseases or pathological conditions which are associated with nitric oxide levels in cells and tissue such that inhibition of NOS to decrease nitric oxide levels ameliorates the disease or condition.

The invention further provides methods for treatment of diseases or pathological conditions associated with calmodulin activity in cells or tissues such that decreasing the activity of calmodulin in these cells or tissues results in an amelioration of the disease or condition. The methods of treatment of this invention can generally be applied to any individual in need of treatment including mammals and including humans. The compounds herein have application to human and/or veterinary treatment.

In addition, the water-soluble fullerenes of this invention have non-therapeutic applications as research reagents for the study of biological systems.

### DETAILED DESCRIPTION OF THE INVENTION

The present invention relates generally to fullerene derivatives that have increased solubility in water and aqueous solutions compared to non-derivatized fullerenes.

The term “fullerene” is used generally to refer to any closed cage carbon compound containing both six-and five-member carbon rings independent of size and is intended to include the abundant lower molecular weight C₆₀ and C₇₀ fullerenes, larger known fullerenes including C₇₆, C₇₈, C₈₄ and higher molecular weight fullerenes C_{2N} where N is 50 or more. The term is generally intended to include “solvent extractable fullerenes” as that term is understood in the art (generally including the lower molecular weight fullerenes that are soluble in toluene or xylene). While the term fullerene also includes higher molecular weight fullerenes that cannot be extracted, including giant fullerenes which can be at least as large as C₄₀₀, these forms of fullerenes are not preferred for use in this invention. Preferred fullerene for formation of fullerene derivatives of this invention are C₆₀ and C₇₀ fullerenes. Fullerenes for use in this invention can be prepared by any known method. Combustion methods which currently are the most efficient methods for fullerene production are the preferred method for producing fullerenes of this invention. It is also preferred that fullerenes of this invention are prepared and/or purified by methods that minimize or eliminate polyaromatic hydrocarbons (PAHs) in the product fullerene.

The fullerene derivatives of this invention exhibit measurable solubility in water and have increased solubility in water compared to the non-derivatized fullerene. Water-solubility for purposes of this invention is determined as described in C. F. Richardson et al. (2000) Org. Lett. 2(8):1011-1014 in which saturated solutions of the derivatized fullerene are prepared by stirring in water for 36 h, followed by centrifugation to remove any suspended and/or non-dissolved material, and measurement of UV absorption at 257 nm. Preferred water-soluble fullerenes have solubility in water at room temperature of 100 μM or more. More preferred water-soluble fullerenes have solubility in water at room temperature of 500 μM or more.

Water-soluble fullerene derivatives of this invention have at least one water solubilizing substituent covalently attached to the fullerene cage. Water-solubilizing substituents are generally any groups that are hydrophillic in character and may be polar or contain a polar moiety, be charged or contain a charged moiety (anionic or cationic), or be zwitterionic or contain a zwitterionic moiety (a zwitterionic group or moiety contains a negatively charged species and a positively charged species, but is charge neutral).

Water-soluble fullerenes of this invention include fullerenes of Formula I:

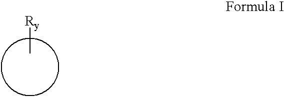

containing at least one water-solubilizing substituent R, e.g., a substituent that is a charged, zwitterionic, polar or other hydrophillic group. In general, however, R of Formula I can be selected from the group of substituents that are either hydrophobic or hydrophilic so long as the derivatized fullerene is water-soluble to some extent. In preferred embodiments all substituents on the fullerene are hydrophillic.

More specifically R, independent of other R, can be selected from the group consisting of —H, —OH, —SH, —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(R₃), —O C(R₁)(R₂)(R₃), -halide (e.g., —F, —Cl, —Br), —(CH₂)ₙ—N(R₄)₂, —O—(CH₂)ₙ—N(R₄)₂, —NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺, —(CH₂)ₙ—OH, —O—(CH₂)ₙ—OH, —NH—(CH₂)ₙ—OH, —C(O)OR₁, —C(O)N(R₁)(R₂), alkyl groups, alkoxy groups, thioakyl groups (—SR), aryl groups or aryloxy groups (—O-aryl) optionally substituted with one or more of —NH₂, —NO₂, —OH, —CH₂—OH, —(CH₂—O)ₙX₁, -halide, akylhalide, —N(R₄)₂, —N(R₄)₃⁺, —COOH, —COO⁻, —SO₃⁻, —SO₃H, —PO(OR₄)₂, —PO₂OR⁻, or —PO₃²⁻ wherein n is an integer ranging from 1 to about 200 (including n=1-100, n=1-20, n=1-10 and n=1-6), where R₁₋₃, independently, are selected from the group consisting of —H, —OH, —NH₂, -optionally substituted alkyl (preferably lower alkyl with 1-6 carbons, including methyl and ethyl), optionally substituted aryl, —SO₃H, —SO₃⁻, —P(O)(OH)₂, —P(O)₂(OH)⁻—COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl (preferably lower alkyl having 1-6 carbon atoms), optionally substituted aryl, —CH₂—OH, —COOH, —COO⁻, —CH₂—N(R₄)₂; X₁ is selected from the groups consisting of, —H, -alkyl (preferably lower alkyl having 1-6 carbon atoms), and —CH₂—OH, wherein any anion in the compound can be paired to an appropriate cation C⁺ with an appropriate charge to achieve charge neutrality and any cation in the compound can be paired to an appropriate anion, A⁻ with an appropriate charge to achieve charge neutrality. Preferred anions and cations are those that are pharmaceutically acceptable as is recognized in the art. Anions include halides, organic acid anions, acetate, trifluoroacetate, etc. Cations include alkali and alkaline earth metal cations, etc. In addition two or more or the R groups on the fullerene may be linked together to form a bridging substituent that bridges two or more carbons on the fullerene. Bridging R substituents include, among others:

—N(R₁)(R₂)—(CH₂)ₙ—N(R₃)—;

—C(R₁)(R₂)—N(R₅)(R₆)⁺—C(R₁)(R₂)—;

>C(COOR₂)₂,

>C(R₁)(COOR₂);

>C(R₁)(CON(R₂)(R₃));

>C(CON(R₂)(R₃))₂;

>C(P(O)(OR₄)₂)₂,

>C(P(O)(OR₄)₂)(P(O)(OR₄)O⁻;

where the symbol “>” indicates two bonds to the fullerene, R₅ and R₆, independently, can be the same groups as R₄ and all other groups are as defined above.

Charged groups that can be carried on R substituents include among other various phosphorous oxide anions, various sulfur oxide anions, various carbon oxide anions (e.g., carboxylate, carbonate, etc.), various nitrogen cations (e.g., protonated primary, secondary, or tertiary amine cations, quaternary amine cations, etc.). In general charged groups can have any level of charge (e.g., 1+, 2+, 3+ etc. or 1−, 2−, 3−, etc.). Zwitterionic groups can contain any combination of paired positively charged moiety and negatively charged moiety, e.g., —COO⁻0 or —SO₃⁻ paired with —N(R₄)₃⁺.

In addition, fullerene derivatives of this invention include those in which one or more of R₁₋₆ can be an amino acid or a peptide, preferably bonded to the fullerene in such a way that it retains its zwitterionic character. Further, any one or more of R can be protected esters, polyethylene glycols, polyols, and/or water-solubilizing polymers (e.g., polymers carrying a plurality of polar, charged, preferably positively charged, or zwitterionic groups).

In specific embodiments, derivatized fullerenes of this invention have formula II, where y is the number of malonate groups on the fullerene (y is an integer from 1-30 and is preferably 1-6) and Y and Z can be any of the R groups listed above. Either or both of Y and/or Z can be protected esters, polyethylene glycols, polyols, and/or water-solubilizing polymers. In specific preferred embodiments, one or both of Y and Z is a group selected from —O—(CH₂)ₙ—N(R₄)₂, or —O—(CH₂)ₙ—N(R₄)₃⁺A⁻ where n is an integer from 1-20 (preferably 1-10 and more preferably 3-6) R₄ is as defined above and is preferably —H or lower alkyl groups having 1-6 carbon atoms (methyl, ethyl, etc.). If only one of Y and/or Z carries an amine group then the other of Y or Z can generally be selected from any of the groups listed for R above and in particular may be an optionally substituted alkyl or aryl group.

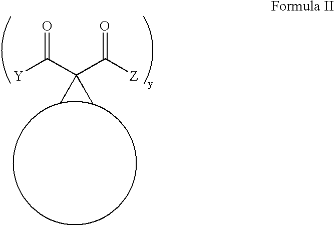

Alkyl groups and the alkyl portion of alkoxy or alkylaryl groups in the definitions herein are saturated (or cyclic) hydrocarbons which can be straight-chain, branched or cyclic and unless otherwise noted can have 1-20 carbons atoms. Cyclic alkyl groups, such as cyclohexane, can be heterocyclic containing one or more (typically one or two heteroatoms, e.g., N or O.) In some cases, lower alkyl groups which contain from 1 to 6 carbon atoms are preferred. Lower alkyl groups are any of the various linear, branched or cyclic alkyl groups having from 1 to 6 carbon atoms. Aryl groups in the definitions herein are groups having one or more aromatic rings. Aryl groups include heteroaromatic groups, such as a pyridine group. Aryl groups include alkylaryl groups in which the aryl group is linked into a structure through an alkyl group or in which the aryl group is substituted with one or more alkyl groups. Aryl groups may contain one or more aromatic rings (e.g., phenyl ring or a pyridine ring) which may be fused (e.g., naphthalene) and in which one or more of the ring carbons can be replaced with N or O, (e.g., pyridine). Aryl groups herein contain 6 or more carbons. Alkyl and aryl groups herein can be optionally substituted with any one or more of the following groups; —OH, -halide, perhalogenated alkyl (—CF₃), halogenated alkyl groups (e.g., —CHF₂), —NO₂, —NH₂, —N(R₄)₂ (where R₄ is as defined above), —N(R₄)⁺₃, —COOH, —COOR₁, and or —COO⁻.

In one aspect fullerene derivatives of this invention inhibit NOS activity. The fullerene derivatives of this invention have IC₅₀ values (the concentration of fullerene derivative which inhibits NOS activity by 50%) lower than 500 μM, preferably have IC₅₀ values of 100 μM or less, more preferably have IC₅₀ values of 10 μM or less, and yet more preferably, values of 2 μM or less. Table 1 summarizes IC₅₀ values for representative fullerene derivatives of this invention.

TABLE 1
Inhibition of NOS Activity by Fullerene Derivatives

| | IC50 for Inhibition of Citrulline | | |
|---|---|---|---|
| | Formation μM | | |
| Fullerene (C₆₀) | iNOS | nNOS | eNOS |
| Mono-malonyl adduct, | 19 | 2.5 | 77 |
| diamine salt | | | |
| D₃ Tris-malonyl adduct | 5.5 | 0.6 | 10 |
| diamine salt | | | |
| Polyhydroxylated fullerene | 40 | 5.4 | 2 |
| Mono-malonyl adduct diol | 110 | 4.0 | 220 |
| Tris-malonyl semi-amine | 1.3 | 0.6 | 16 |
| Mono-malonyl semi-amine | 85 | 11 | 89 |
| C₃-Tris-adduct diamine salt | 17 | 2 | 29 |
| C₃-Tris malonyl adduct diacid | 123 | 24 | 17 |
| D₃-Tris malonyl adduct diacid | 317 | 86 | 143 |
| C₇₀ | 41 | 10 | 29 |

In another embodiment, certain fullerene derivatives of this invention exhibit selective modulation, or more specifically, selective inhibition, of nNOS and/or iNOS compared to eNOS. Selective nNOS or iNOS inhibitors of this invention have IC₅₀, binding or inhibitory parameters which are lower than the corresponding values for eNOS. Most generally, a preferred selective inhibitor or nNOS and/or eNOS exhibits little or no inhibition of iNOS at concentration levels at which it is inhibitory for nNOS or eNOS. More preferred selective inhibitors are those that exhibit the lowest relative inhibition levels of eNOS. Preferably, fullerene iNOS and nNOS inhibitors of this embodiment have IC₅₀ binding or inhibitory parameters which are 2-fold lower, preferably 10-fold lower, more preferably 50-fold lower and yet more preferably 100-fold lower than the respective values for eNOS.

Certain fullerene derivatives herein and experiments concerning NOS inhibition and calmodulin inhibition by fullerene derivatives have been reported in D. J. Wolff et al. (2000) Arch. Biochem. Biophys. 378(2) (June) 216-223; C. F. Richardson et al. (2000) Org. Lett. 2(8):1011-1014; Wolff, D. J et al. (2001) Biochemistry 40:37-45; and D. J. Wolff et al. (2002) 2(March):130-141. These references are incorporated by reference herein in their entirety and specifically for their teachings with respect to synthesis and characterization of fullerene derivatives and experimental results regarding NOS inhibition and calmodulin inhibition exhibited by fullerene derivatives. These references contain specific discussion of the experimental details concerning various assays performed using water-soluble fullerenes of this invention.

C₃-tris-malonyl-C₆₀-fullerene and D₃-tris-malonyl-C₆₀-fullerene derivatives inhibit citrulline and NO formation by all three nitric oxide synthase isoforms in a manner that is fully reversible by dilution. The inhibition of citrulline formation by C₃-tris-malonyl-C₆₀-fullerene occurs with IC₅₀ values of 24, 17, and 123 μM for the neuronal, endothelial, and inducible nitric oxide synthase (NOS) isoforms, respectively (see Table 1). As measured at 100 mM L-arginine, nNOS-catalyzed nitric oxide formation was inhibited 50% at a concentration of 25 mM C₃-tris-malonyl-C₆₀-fullerene. This inhibition was a multisite, positively cooperative inhibition with a Hill coefficient of 2.0. C₃-tris-malonyl-C₆₀-fullerene inhibited the arginine-independent NADPH-oxidase activity of nNOS with an IC₅₀ value of 22 μM but had no effects on its cytochrome c reductase activity at concentrations as high as 300 μM. The inhibition of nNOS activity by C₃-tris-malonyl-C₆₀-fullerene reduced the maximal velocity of product formation but did not alter the EC₅₀ value for activation by calmodulin. C₃-tris-malonyl-C₆₀-fullerene reduced the maximal velocity of citrulline formation by iNOS without altering the Km for L-arginine substrate or the EC₅₀ value for tetrahydrobiopterin co-factor. As measured by sucrose density gradient centrifugation, fully inhibitory concentrations of C₃-tris-malonyl-C₆₀-fullerene did not produce a dissociation of nNOS dimers into monomers. These observations indicate that C₃-tris-malonyl-C₆₀-fullerene inhibits the inter-subunit transfer of electrons. Although not wishing to be bound by any particular mechanism the inhibition observed may be mediated by a reversible distortion of the dimer interface.

C₃- and D₃-tris-malonyl-C₆₀-fullerenes inhibit all three nitric oxide synthase isoforms. The inhibition by the fullerenes of the Ca²⁺ and CaM-dependent constitutive nNOS and eNOS isoforms was more potent than that observed for the cytokine-inducible isoform (Table I). The C₃ isomer was a more potent inhibitor of all three NOS isoforms than was the D₃ isomer. In considering the differences observed between the two regioisomers, the C₃ isomer exhibits a much more exposed hydrophobic “surface,” having three bridges carrying the polar groups gathered closer together in space, in contrast with the D₃ isomer where the polar groups are almost equally distributed over the spherical surface of the C₆₀ shell. The interaction between NOS and fullerene may involve this hydrophobic face. The inhibition of NOS activity by C₃-tris-malonyl-C₆₀-fullerene is apparently not exerted at the arginine substrate site, since inhibition versus arginine was non-competitive. The inhibition by these fullerenes is also not attributable to competition with BH₄ for its stimulatory site on NOS, since the inhibition was in-surmountable by increased concentrations of BH₄. Further, both C₃- and D₃-tris-malonyl-C₆₀-fullerenes inhibited the arginine-independent NADPH-oxidase activity of nNOS, an activity that is independent of either arginine substrate or BH₄ cofactor.

C₆₀-fullerene mono-malonyl adducts selectively inactive or inhibit nNOS isoform in a manner that is completely preventable by the concurrent presence of superoxide dismutase and catalase. Of the fullerenes tested, the diol adduct exhibited the greatest isoform selectivity, inhibiting the neuronal isoform at concentrations 55-fold lower than those required to inhibit the endothelial isoform and 28-fold lower than those required to inhibit the cytokine-inducible isoform. Inhibition of nNOS is time-dependent, fullerene concentration-dependent, and turnover-dependent and is not reversible by dilution. In contrast, the inhibition of iNOS and eNOS by these fullerene adducts appears to be reversible. The mono-adduct diol has no effect on NADPH consumption by nNOS as measured in the absence of arginine substrate, but significantly increases the consumption of NADPH in the presence of arginine. This fullerene-enhanced NADPH consumption is linked to oxygen as electron acceptor and is accompanied by the increased production of hydrogen peroxide. These effects of the mono malonate adducts are unique to the nNOS isoform and are not observed using either the iNOS or eNOS isoforms. The inhibitory effects of fullerene monomalonate adducts are unaltered and insurmountable by increased concentrations of arginine, tertahydrobiopterin, or calmodulin. Again without wishing to be bound by any particular mechanism of action, these results indicate that fullerene monomalonate adducts which exhibit selective inhibition of nNOS function to uncouple the formation of reactive oxygen intermediates from NO production in the presence of arginine. Reactive oxygen intermediates dissociate from the enzyme and acting from solution, inactive NOS NO forming ability. It is of interest to note that the fullerene mono adducts each possess a large spherical hydrophobic domain in contrast to the tris adducts in which the hydrophillic groups are distributed around the fullerene sphere. The presence of this large hydrophobic domain may be at least in part responsible for the selective inhibition exhibited by these fullerenes.

C₆₀-Fullerene tris adducts carrying amine groups inhibit nNOS and calcineurin phosphatase activities in a manner completely reversible by calmodulin. As measured by difference spectroscopy, D₃-trisamine and C₃-semiamine fullerene adducts displace trifluoperazine bound to calmodulin coincident with their binding. These binding events are complete at a molar ratio of 4 mol added fullerene per mole calmodulin. Trisamine fullerene adducts alter the native electrophoretic mobility of calmodulin, producing a heterogeneity of bands with associated fullerene. D₃- and C₃-trisamine fullerene adducts interact with dansylated calmodulin, producing a 50% loss of maximal fluorescence at concentrations of 30 nM. At higher concentrations than those required to inhibit nNOS, trisamine fullerene adducts inhibit nitric oxide formation by the cytokine-inducible nitric oxide synthase isoform (iNOS). These inhibitions are fully reversible by calmodulin and skeletal muscle troponin C, but not by skeletal muscle parvalbumin. In addition, the C₃- and D₃-semiamine adducts were found capable of inhibiting Ca²⁺-dependent nitric oxide production in GH3 pituitary cells. The trisamine C₆₀-fullerene adducts are potent calmodulin antagonists as shown in Table 2.

TABLE 2
Determination of the IC₅₀ Values for Diverse CaM
Antagonists in Inhibiting Citrulline Formation by nNOS^{a}

| Compound | IC₅₀ value, nM |
|---|---|
| C₃-trisamine | 63 |
| D₃-trisamine | 38 |
| C₃-tris-semiamine | 140 |
| D₃-tris-semiamine | 114 |
| C₃-mixed ester | 231 |
| D₃-mixed ester | 615 |
| CBP | 3 |
| CIP | 23 |
| Calmidazolium | 273 |
| Trifluoperazine | 6600 |
| ^{a}Standard incubations were constructed for measurement of citrulline formation in incubations containing 6 nM CaM as described under Experimental Procedures in the absence and presence of concentrations of fullerene adducts ranging from 1 nM to 300 μM. The concentration of agent providing a 50% inhibition of activity in a 30-min incubation is indicated. Incubations were initiated with an nNOS dilution that provided less than 10% consumption of substrate as measured in the | |
| #absence of added agent. | |

The fullerene derivatives of this invention are readily synthesized employing methods provided herein in view of methods that are well-known in the art with regard to the introduction of substituents onto the fullerene cage. General techniques of synthetic organic chemistry can be readily applied by one of ordinary skill in the art in view of the teachings herein and what is known in the art to prepare any of the fullerene derivatives of this invention. In specific embodiments herein Bingle-Hirsch addition reactions have been used to produce malonyl adducts of fullerenes. This methodology is convenient and preferred for ease of application in making certain derivatives, but other methods for derivatization of fullerenes are known in the art and can be applied to the synthesis of the fullerene derivatives of this invention. A variety of procedures for functionalization of fullerenes are available.

The methods described in the following references can be employed in the synthesis of the fullerene derivatives of this invention: Fullerenes Synthesis, Properties, and Chemistry of Large Carbon Clusters, G. Hammond, et al., Eds., ACS Symposium Series 481, American Chemical Society, Washington, D.C., 1992; see entire issue No. 3 of Acc. Chem. Res., 25, 1992; A. Hirsch, et al., Chem. Int. Ed. Engl., 31:766, 1992; and U.S. Pat. Nos. 6,204,391; 6,162,926; 6,046,361; 5,994,410; Hirsch, A. (1994), “Cycloadditions” in The Chemistry of Fullerenes, Thieme Medical Publishers, New York 99 79-112; Hirsch, A. (1999), Principles of Fullerene Reactivity in Topics in Current Chemistry: Fullerenes and Related Structures, Vol. 199, Springer-Verlag, Berlin, New York, pp. 2-60; Prato, M. and Maggini, M. (1998), “Fullereopyrollidines: A Family of Full-Fledged Fullerene Derivatives,” Acc. Chem. Res. 31:519-526; Tomberli, V. et al. (2000), “Synthetic Approaches to the Preparation of Water-Soluble Fulleropyrollidines,” Carbon 38:1551-1555; Grosser, T. et al. (1995) “Ring Expansion of the Fullerene Core by Highly Regioselective Formation of Diazaftilleriods,” Ang. Chemie. Int. Ed. Eng. 34:1343-1345; Ulmer, L. et al. (1998), “Mono- and bisfunctionalization of fullerenes with N-containing reactants,” J. Inf. Rec. 24(3-4):243-247; Sun et al. (1997), “Photochemical preparation of highly water-soluble pendant (60) fullerene-aminopolymers,” Photochem. Photobiol. 66(93):301-308; Illescas, B. et al. (1997), “(60) Fullerene-based electron acceptors with tetracyano-p-quinodimethane (TCNQ) and dicyano-p-quinonediimine (DCNQI) derivatives,” Tetrahedron Lett. 38(11):2015-2018; Sun et al. (1996), “Preparation and characterization of a highly water-soluble pendant fullerene polymer,” Chem. Commun. No. 24:2699-2700; Maggini, M. et al. (1994), “Addition reactions of C₆₀ leading to fulleroprolines,” J. Chem. Soc., Chem. Commun. No. 3:305-306; Bellavia-Lund, C. and Wudl, F. (1997), “Synthesis of (70) Azafulleroids: Investigations of Azide Addition to C₇₀,” J. Am. Chem. Soc. 119(41):9937. All of the preceding references are incorporated by reference herein in their entirety.

This invention relates to certain compounds which inhibit NOS and to certain compounds which inhibit calmodulin. These compounds have therapeutic and non-therapeutic applications. Non-therapeutic applications include their use as research reagents for the study of the biological mechanism(s) of nitric oxide action and the regulatory activity of calmodulin. Inhibition of NOS and/or calmodulin is assessed generally by measuring some biological activity of NOS or calmodulin, respectively. NOS activity and calmodulin activity are preferably assayed as described herein, but can be assessed by any method known in the art.

In one aspect for therapeutic applications the invention provides therapeutic compositions for the treatment of diseases, disorders or pathological conditions that are associated with or attributable to nitric oxide levels in cells or tissues of an individual. These therapeutic compositions comprise a pharmaceutically acceptable carrier and an amount or combined amount of one or more water-soluble fullerene derivatives of this invention or pharmaceutically acceptable salts thereof effective for inhibition of NOS. In specific embodiments, pharmaceutical compositions herein comprise a pharmaceutically acceptable carrier and one or more water-soluble fullerene derivatives of this invention or pharmaceutically acceptable salts thereof which exhibit selective inhibition of nNOS or iNOS rather than eNOS. In these compositions the fullerene derivative selective inhibitor(s) are present in an amount or in a combined amount effective for exhibiting selective inhibition of nNOS and/or iNOS rather than eNOS.

In another aspect for therapeutic applications, the invention provides therapeutic compositions for the treatment of diseases, disorders or pathological conditions that are associated with or attributable to calmodulin activity in cells or tissues of an individual. These therapeutic compositions comprise a pharmaceutically acceptable carrier and an amount or combined amount of one or more water-soluble fullerene derivatives of this invention or pharmaceutically acceptable salts thereof effective for inhibition of calmodulin. In specific embodiments, pharmaceutical compositions herein comprise a pharmaceutically acceptable carrier and one or more water-soluble fullerene derivatives of this invention or pharmaceutically acceptable salts thereof.

The term “effective amount” of fullerene derivatives of this invention is the amount or combined amount (if more than one fullerene derivative is combined) that provides a measurable improvement in the symptoms of the disease, disorder or condition that is to be treated. Improvements resulting from treatment are to be assessed by standard clinical methods by one of ordinary skill in the art and these improvements should be clinically significant. The treatment may cure the disease, disorder or condition or provide relief or remission of the symptoms of the disease, disorder or condition. The term “ameliorate” is used to encompass all of these outcomes of treatment.

The effective amount appropriate for treatment of a given disease, disorder of condition is determined empirically based on the severity of the disease, disorder or condition, the age, weight, health status and condition of the individual (e.g., human patient) to be treated, the treatment schedule and the desired outcome of treatment. The determination of effective amounts of medicaments is generally within the skill of one of ordinary skill in the art and can be determined without undue experimentation. Most generally, the therapeutic compositions of this invention will contain from about 0.1% to 99.99% by weight of one or more active ingredients. More typically, therapeutic compositions of this invention will contain from about 1% by weight to about 99% by weight of one or more active ingredients. Therapeutic compositions of this invention, dependent upon the application and mode of delivery, can include active ingredients, beneficial for treatment of the disease, disorder, or condition, in addition to the fullerene derivatives herein.

Those of ordinary skill in the art will appreciate that the compositional details of therapeutic compositions of this invention can be determined using appropriate animal models or other appropriate routine experimentation known in the art.

The therapeutic compositions of this invention can take a variety of dosage forms for administration by various routes. In particular, therapeutic compositions can be readily prepared for oral, topical, infusion, intravenous, intraperitoneal, subcutaneous, or intramuscular administration or for administration via aerosol or solution spray or suppositories. The compositions of this inventions can be formulated in a form appropriate for the disease, disorder or condition that is to be treated and the chosen mode of administration, for example, in a solid, semi-solid, or liquid dosage form, including among others, tablets, pills, powders, liquid solutions or suspensions, emulsions, creams, suppositories, injectable and infusible solutions and nasal sprays.

The therapeutic compositions of this invention can be formulated by those of ordinary skill in the art employing known methods for preparing pharmaceutically useful compositions. A number of reference sources are known and readily available to those skilled in the art for preparation of formulations. For example, the teachings of Remington's Pharmaceutical Science by E. W. Martin describing pharmaceutical formulation can be employed or readily adapted in view of other techniques that are well-known in the art to the preparation of therapeutic compositions of this invention.

The therapeutic compositions of this invention employ pharmaceutically acceptable carriers. Such carriers are well-known in the art and appropriate carriers can be readily chosen in view of the form of administration, the nature and amount of the active ingredient to be incorporated and for compatibility with the active ingredients to be included.

Certain of the compounds of this invention have limited water-solubility and as such formulation methods that take into account this limited solubility can be beneficially employed in the formulation of therapeutic compositions of this invention. For example, the use of emulsion formulations, liposome formulations or the use of additives or methods of formulation that facilitate or enhance solubility of active ingredients in aqueous solutions can be readily employed or adapted. Where appropriate for the contemplated treatment, slow- or controlled-release methods of administration of the therapeutic compositions employing capsules, implanted pumps, or biodegradable carriers or containers can be applied.

The invention is further illustrated in the following non-limiting examples.

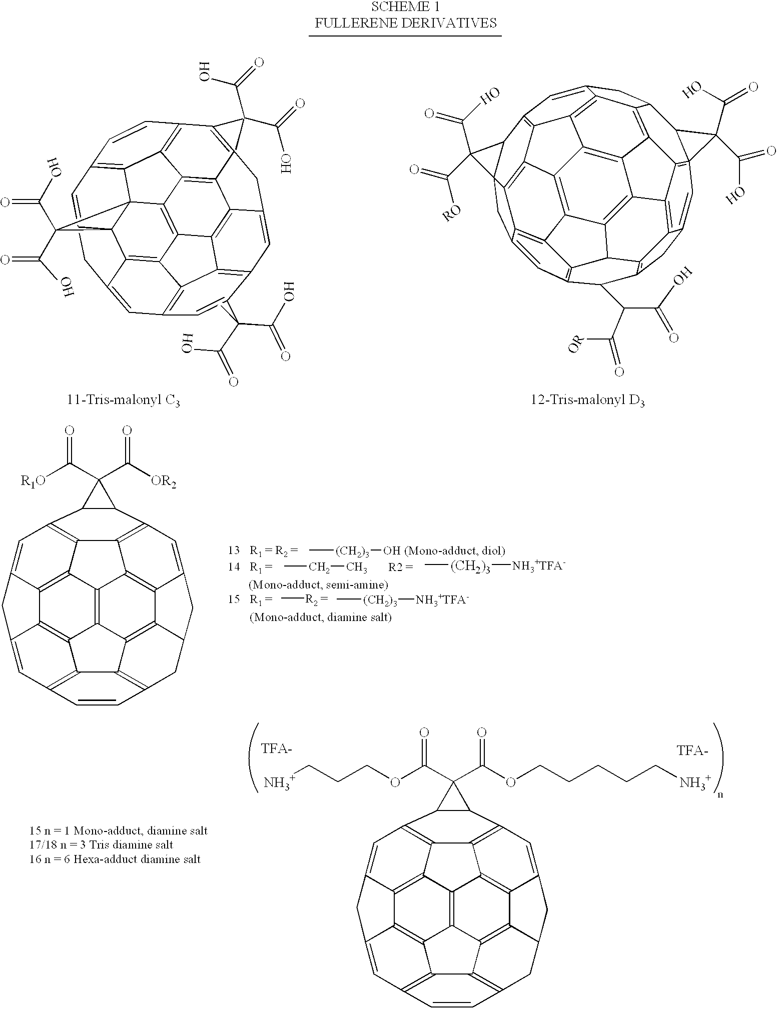

### EXAMPLES

### Example 1

### Synthesis of C₃ and D₃-tris-malonic Ester Adducts and Free Acids

Synthesis and Chromatographic Resolution of the C₃- and D₃-tris-malonyl Ethyl Esters.

C₃-tris-[methano-di(ethoxycarbonyl)]-[60]fullerene (C₃-tris-malonyl-C₆₀-fullerene ethyl ester) and D₃-tris-[methano-di(ethoxycarbonyl)][60]fullerene (D₃-tris-malonyl-C₆₀-fullerene ethyl ester) were synthesized (with a minor modification) by the procedure of Hirsch et al. (Hirsch, A. et al. (1994) J. Amer. Chem. Soc. 116:9385-9386; Hirsch et al. (1994) Angew. Chem. Int. Ed. Eng. 33:437-438). Briefly, to a solution of 500 mg (1 eq) of C₆₀ fullerene dissolved in 300 mL of toluene by sonication was added 0.62 mL (6.6 eq) of 1,8-diazabicyclo[5.4.0]undec-7-ene (DBU). Diethyl-bromomalonate (0.36 mL, 3.3 eq) was added in three portions with 5 min between each addition. Following the last addition, the reaction mixture was immediately quenched with 100 mL of water. After washing with water (2 3 100 mL), evaporation, and drying over sodium sulfate, the crude reaction mixture was separated on a silica gel column using the following eluents: toluene; toluene/ethyl acetate 1000:1, toluene/ethyl acetate 100:1. The tris-substituted isomers eluted sequentially in the toluene/ethyl acetate 100:1 eluent.

Synthesis of Free Acids

1,2:18,36:31,32-Tris(methano)[60] fullerene-61, 61, 62, 62, 63, 63 hexa-carboxylic acid, (e,e,e) C₃ symmetry isomer (C₃-tris-maloyl, C₆₀-fullerene) and 1,2:34,35:40,41-tris(methano)[60] fullerene-61, 61, 62, 62, 63, 63 hexacarboxylic acid, (trans-3, trans-3, trans-3) D₃ symmetry isomer (D₃-tris-malonyl-C₆₀-fullerene) were synthesized as described by Lamparth and Hirsch (Hirsch, A. et al. (1994) Angew. Chem. Int. Ed. Engl. 33:437-438, for structures see Scheme 1). To a solution of 100 mg (1 eq) of ester was added 115 mg (60 eq) of NaH. The mixture was allowed to reflux for 1 h at which time 5 mL of methanol was added. A solid precipitated and the clear solution was filtered on a Buchner funnel and was washed with toluene (2×5 mL) and hexane (2×5 mL). The solid was then dissolved in water and was precipitated by the addition of 3 M HCl. After filtration the precipitate was washed with 3 M HCl (5 mL) and water (2×5 mL) and then dissolved in methanol. After evaporation and drying, 68.7 mg (83.7%) of acid was obtained. The structure of C₃ and D₃ isomers was confirmed by ¹H and ¹³C NMR spectroscopy, the spectra obtained being identical to those described previously for these regioisomers.

### Example 2

### Synthesis of Amino-Substituted Malonate Derivatives of Fullerenes

A series of derivatives containing an increasing number of malonate appendages with polar groups on the ends were investigated. The reliable Bingel-Hirsch addition of malonates to C₆₀ was used since it allowed the introduction of two polar headgroups for every malonate attached. The malonate reagent:

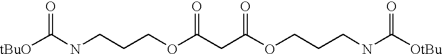

was prepared by treatment of 5 g (2 eq) of tert-butyl N-3(3-hydroxypropyl) carbamate in 250 mL of dry methylene chloride with 1.97 g (1 eq) of distilled malonyl chloride and 2.21 g (2 eq) of pyridine. Following purification by column chromatography on silica gel using 1:1 hexane/ethyl acetate as the eluent, 3.20 g (54.7%) of the illustrated malonate was obtained.

Preparation of the C₆₀ mono adduct of the protected malonate reagent proceeded smoothly according to the general procedure of Hirsch and co-workers (Camps, X and Hirsch, A. J. (1997) J. Chem. Soc., Perkin Trans. 1:1595-1596). To that end, 500 mg (1 eq) of C₆₀ and 331 mg (1.5 eq) of CBr₄ were dissolved in 300 mL of toluene by sonication, followed by the addition of 418 mg (1.5 eq) of the malonate reagent (above) and 0.3 mL (3 eq) of DBU. The reaction was complete in 30 minutes. After conventional workup, the crude reaction mixture was chromatographed on a silica gel column using toluene as eluent to remove unreacted C₆₀, followed by 10:1 toluene/ethyl acetate to yield, after evaporation and drying, 334.8 mg (51.8%) of the desired mono adduct. The carbamate protecting groups were removed by treatment with trifluoroacetic acid to yield 15 (Scheme 1). Although the addition of two amino groups did not appear superficially to confer significant water solubility, a saturated solution of 15 was prepared by allowing the compound to stir in water for 36 h. Afterward, the solution was centrifuged and a sample analyzed by UV spectroscopy at 257 nm; based on the absorbance, the solubility of 15 is 0.64 mg/mL.

The number of malonate substituents connected to the fullerene core was then increased to three by using 3.3 eq of CBr₄, 3.3 eq of the malonate reagent, and 6.6 eq of DBU. After workup, the crude residue was chromatographed on silica gel using 10:1 toluene/ethyl acetate followed by 2:1 toluene: ethyl acetate to give the e,e,e isomer of the tris malonate adduct as a red-orange solid in 10.4% yield (R_{f}-0.25 (silica gel, 2:1 toluene:ethyl acetate); FAB-MS: calcd for C₁₁₇O₄₈N₁₂ 3216, found 3217.9). The UV-VIS absorption spectrum of the tris malonate reagent adduct was identical with that of the e, e, e Hirsch tris-ethyl ester. The carbamate protecting groups were removed using trifluoracetic acid to give the tris-e,e,e adduct 17. To determine the water-solubility of 17, a saturated solution was prepared as above by allowing an excess of 17 to stir in water for 36 h. After centrifugation, analysis by UV spectroscopy at 257 nm indicated that the solubility of 17 in water is 152 mg/mL.

The octahedral (Th) hexa malonate derivative 16 (carrying 12 amines) was synthesized as follows. Malonate reagent (10 eg), 10 eq of CBr₄, and 20 eq of DBU were subjected to the same reaction conditions as above. Following workup, the crude reaction mixture was separated on a silica gel column using 2:1 toluene/ethyl acetate and an orange-yellow band was isolated. This band was then rechromatographed on a preparative TLC plate using 2:1 toluene/ethyl acetate to yield the hexa malonate adduct as a yellow solid R_{f}=0.42 (silica gel, 2:1 ethyl acetate:toluene). In the ¹³C NMR spectrum, only three distinct signals appear at δ 69, 141, and 145 ppm, corresponding, respectively, to the three types of sp³ and sp₂ carbon atoms remaining on the fullerene core. This spectrum compares well with the literature report of the analogous Tₕ-symmetrical hexaadduct of diethylmalonate to C₆₀ reported by Hirsch. Further evidence supporting the octahedral symmetry of the hexa malonate adduct is its UV spectrum which is in excellent agreement with that reported for the Hirsch hexaadduct. Upon deprotection, the dodecaamine salt 16 was isolated. Using the same procedure as described above for 17, the solubility of 16 was found to be 418 mg/mL, the highest solubility of any fullerene derivative reported to date.

### Example 3

### Synthesis of the Fullerene Mono Adduct Diol

Synthesis of Di[3-(tert-butyldimethylsilanyloxy)propyl] malonate.

To a solution of 5 g (0.03 mol) of 3-(tert-butyldimethylsilanyloxy)-1-propanol in 200 mL of dry methylene chloride under nitrogen was added 2.08 g (0.03 mol) of pyridine. To this was added 1.85 g (0.01 mol) of freshly distilled malonyl dichloride. The resulting solution was allowed to stir at room temperature for 2 h, at which time the reaction was 4 stopped. Then 100 mL of saturated sodium bicarbonate was added, and the mixture was extracted into 3×100 mL of ether. The ether extracts were combined, dried over anhydrous sodium sulfate, and evaporated. The crude reaction mixture was separated on a silica gel column using 25% ethyl ether/hexane as the eluent (% yield 49%). Rf=0.6, silica gel, 25% ethyl ether/hexane.

Coupling of Di[3-(tert-butyldimethylsilanyloxy)propyl]-malonate to C₆₀-Fullerene

To a solution of 1 g (1 equiv) of C₆₀ and 691 mg (1.5 equiv) of carbon tetrabromide (CBr₄) in 500 mL of toluene was added 933 mg (1.5 equiv) of di-[3-(tert-butyldimethylsilanyloxy)propyl]malonate followed by 634 mg (3 equiv) of 1,8-diazobicyclo[5.4.0]undec-7-3n3 (DBU). The mixture was allowed to stir for 30 min, at which time the product was observed on TLC. The crude reaction mixture was washed with water (3×100 mL), dried over anhydrous sodium sulfate, and evaporated. The product was separated on a silica gel column using 1:1 hexane/toluene as eluent to remove unreacted C_{b60} followed by toluene to elute the monoadduct (% yield based on recovered C₆₀ 47.3%). R_{f}=0.64, silica gel, 100:1 toluene/ethyl acetate.

### Example 4

### Synthesis of the Semi-Amine Fullerene Adduct (14)

Synthesis of [Ethyl-3-tert-butoxycarbonylaminopropyl]malonate

To a solution of 5 g (0.03 mol) of tert-butyl N-(3-hydroxypropyl)carbamate in 200 mL of dry methylene chloride under an inert atmosphere was added 2.29 g (0.03 mol) of pyridine. To this was added 4.36 g (0.03 mol) of ethyl malonyl chloride. The mixture was allowed to stir overnight. Then 100 mL of saturated sodium bicarbonate was added, and the mixture was extracted with 3×100 mL of diethyl ether. The ether extracts were combined, dried over anhydrous sodium sulfate, and evaporated. The mixture was separated on a silica gel column using 25% ethyl ether/hexane as eluent (% yield 84%).

Coupling of [Ethyl-3-tert-butoxycarbonylaminopropyl]-malonate to C₆₀-Fullerene

To a solution of 5 g (1 equiv) of C₆₀ and 3.45 g (1.5 equiv) of CBr₄ in 3 L of toluene was added 3 g (1.5 equiv) of malonate followed by 3.1 g (3 equiv) of DBU. The mixture was allowed to stir at room temperature for 30 min, at which time the product was visible on TLC. The reaction mixture was washed in two portions of approximately 1.5 L each with water (3×1 L), dried over anhydrous sodium sulfate, and evaporated. Each portion was separated on a silica gel column using toluene to remove unreacted C₆₀ and 10:1 toluene/ethyl acetate to remove the product (% yield based on recovered C₆₀ 73.1%; R_{f}=0.56, silica gel, 10:1 toluene/ethyl acetate).

### Example 5

### Synthesis of Tris Diamine, Semiamine and Mixed Ester Fullerenes

Following general procedures from Hirsch and co-workers (Camps, X. and Hirsch, A. J. (1997) J. Chem. Soc., Perkin. Trans. 1:1595-1596) six trismalonate adducts of C₆₀ were prepared (for structures 17-22, see Scheme 1). Fullerene adducts were prepared using a three-step synthetic scheme. Initially malonate esters containing appropriately protected amine residues were prepared. These malonate esters were then coupled either to underivatized C₆₀-fullerene or to C₆₀ singly derivatized with diethylmalonate (mono Maloney adduct). The coupled amino fullerenes were subsequently deprotected by stirring for 30 min in toluene:trifluoroacetic acid 1:1 (Richardson, C. J. et al (2000) Org. Lett.2:1011-1014).

Synthesis of e,e,e- and t,t,t-tris[di-(3-tert-butyloxycarbonylamino-propyl)]methano-[60]-fullerene.(17 and 18)

To a solution of 100 mg (1 eq) of C₆₀ and 151 mg (3.3 eq) of CBr₄ in 60 ml of toluene was added 190 mg (3.3 eq) of di-[3-(tert-butyloxycarbonylaminopropyl)] malonate followed by 138 mg (6 eq) of 1,8-diazobicyclo[5,4,0]undec-7-ene (DBU). The di-[3-(tert-butyloxycarbonylaminopropyl)] malonate was prepared as described above. The reaction was allowed to stir at room temperature and was monitored by thin-layer chromatography (TLC). After 30 min, the color of the reaction changed from purple to a bright red-orange and the reaction was stopped. The reaction mixture was washed with 3×30 ml of water, and the organic layer was dried over sodium sulfate and evaporated. The crude product was separated by sequential elution from a silica gel column using toluene followed by 2:1 toluene:ethyl acetate to remove the products. After evaporation and drying 19.6 mg (10.4%) of tris e,e,e isomer was obtained. The D3 isomer was also isolated but in lower yield (<10%). The C₃ and D₃ regioisomers (compounds 17 and 18) were generated by deprotection with trifluoroacetic acid.

Synthesis of e,e,e- and t,t,t-tris[ethyl-3-tert-butyloxycarbonylamino-propyl]methano-[60]-fullerene.

To a solution of 0.5 g (1 eq) of C₆₀ and 0.759 g (3.3 eq) of CBr₄ dissolved in 300 ml of toluene by sonication was added 0.664 g (3.3 eq) of [ethyl-3-tert-butyloxycarbonylaminopropyl]malonate followed by 0.622 ml (6 eq) of DBU. The [ethyl-3-tert-butyloxycarbonylaminopropyl] malonate was prepared as described above. The mixture was allowed to stir at room temperature for 30 min at which time the color of the reaction mixture changed from purple to red-orange and the products were visible on TLC. The crude mixture was washed with 3×150 ml of water, and the organic layer was dried over anhydrous sodium sulfate and evaporated. The crude mixture was separated on a silica gel column using toluene, 10:1 toluene:ethyl acetate, and 10:3 toluene:ethyl acetate to yield the C₃ and D₃ isomers. After evaporation and drying 10% of both the C₃ and the D₃ isomers was obtained.

Synthesis of e,e,e- and t,t,t-tris[diethylmalonyl-3-tert-butyloxycarbonylaminopropyl]methano-[60]-fullerene.

The starting material for this synthesis was diethyl-C₆₀ monomalonate rather than undervatizedC₆₀-fullerene. Diethyl-C₆₀ monomalonate was prepared as described above. To a solution of 0.1 g (1 eq) of diethyl-C₆₀ monomalonate and 0.075 g (2 eq) of CBr₄ dissolved in 60 mL of toluene by sonication was added 0.095 g (2 eq) of di-[3-(tert-butyloxycarbonylaminopropyl)]malonate and 0.069 g (4 eq) of DBU. The mixture was allowed to stir for 30 min at which time the product was visible on TLC and the reaction was stopped. The color of the reaction mixture changed during the reaction from purple to red-brown. The reaction mixture was washed with 3×50 ml of water and dried over sodium sulfate and evaporated. The products were eluted sequentially from a silica gel column using 2:1 toluene: ethyl acetate as the eluent. After evaporation and drying the e,e,e isomer was obtained as a red solid and the t,t,t isomer was obtained as a brown solid. Yields comparable to the previous tris isomers were observed (10%).

### Example 6

### Biological Assays

Materials.

L-[2,3-H]-Arginine was purchased from New England Nuclear. Murine (interferon-γ) was obtained from Genzyme. BH₄ was purchased from B. Schircks Laboratories (Jona, Switzerland). Hydrogen peroxide, ferrous ammonium sulfate and sodium thiocyanate were obtained from Sigma. Bovine brain calmodulin was prepared by the procedure of Gopalakrisna, R. and Anderson, W. B.(1982) Biochem. Biophys. Res. Commun. 104:830-836. Rabbit skeletal muscle troponin C was prepared by the procedure of Greaser, M. L. and Gergely, J. (1977) J. Biol. Chem. 246:4226-4233. Rabbit skeletal muscle parvalbumin was prepared by the procedure of Capony et al.(1976) Eur. J. Biochem.70:123-135. The calmodulin antagonist peptides calmodulin binding domain (CBP; CaM kinase II residues 290-309), calmodulin inhibitory peptide (CIP; a 17-residue peptide based on the calmodulin binding domain of myosin light chain kinase), and calmodulin inhibitory peptide control were obtained from Calbiochem. The calmodulin antagonist calmidazolium was similarly obtained from Calbiochem. All other reagents were obtained as described by Cooper, G. R. et al. (1998) Arch. Biochem. Biophys. 357:195-206.

Purification of Neuronal NOS.

Neuronal NOS was purified from rat GH₃ pituitary cells grown in Ham's F-10 supplemented with 12.5% horse serum and 3% fetal bovine serum. Cells were homogenized in 5 volumes of 50 mM Mops, pH 7.4, 1 mM EGTA, 1 mM DTT, 100 mM NaCl, 0.5% CHAPSO, 50 μM BH₄, 100 μM leupeptin, and 10 μM E64. A postmitochondrial supernatant fraction was generated by centrifugation and was affinity-purified on 2′,5′ADP-agarose as reported previously (Wolff, D. J. and Datto, G. (1992) Biochem. J. 284:201-206). The affinity-purified proteins were eluted with 50 mM Mops, pH 7.4, 1 mM EGTA, 1 mM DTT, 5 mM NADPH, 0.1% CHAPSO, 150 mM NaCl, 50 μM BH₄, 100 μM leupeptin, and 10 μM E64. The eluate was adjusted to contain 2.5 mM Ca²⁺ and was adsorbed to a calmodulin-Affi Gel 15 agarose column equilibrated in 50 mM Mops, 1 mM EGTA, 2.5 mM Ca²⁺ 0.1% CHAPSO, 150 mM NaCl, 25 μM arginine, 1 mM DTT, 50 μM BH₄, 100 μM leupeptin, and 10 μM E64. The column was washed with equilibration buffer containing 500 mM NaCl but without arginine, and nNOS was eluted with equilibration buffer containing 2.5 mM EGTA but without Ca²⁺ or arginine. Fractions containing nNOS as determined by citrulline formation assay were concentrated with a Centriprep 30 (Amicon). Purified nNOS displayed a single band on a Coomassie blue-stained SDS-PAGE and catalyzed formation of 0.47 μmol citrulline min⁻¹(mg protein)⁻¹ as determined at saturating L-arginine and BH₄ concentrations.

Purification of iNOS.

Cytokine-inducible NOS was purified from murine RAW 264.7 macrophages grown in DMEM/Hepes, pH 7.5, supplemented with 10% fetal bovine serum. Expression of iNOS was induced with 5 μg/mL of Escherichia coli lipopolysaccharide and 50 units/mL of mouse γ-interferon. Homogeneous preparation of the iNOS was obtained through sequential purification on 2′,5′-ADP-agarose and DEAE-cellulose as described previously (Bryk, R. and Wolff, R. (1998) Biochemistry 37:4844-4852). Purified iNOS was about 95% pure as judged by a Coomassie-stained SDS-PAGE.

Purification of eNOS.

Bovine pulmonary arterial endothelial NOS (ENOS) was prepared and characterized as described previously (Wolff, D. J. et al. (1994) Arch. Biochem. Biophys. 312:360-366). These preparations of endothelial NOS routinely possessed a specific activity of 0.15-0.2 mmol citrulline formed/min⁻¹(mg protein)⁻ when assayed at saturating arginine concentrations.

NO Formation Assay.

Nitric oxide production over time was measured spectrophotometrically at 401 nm by the conversion of oxyhemoglobin to methemoglobin (Noack, E. et al. (1992) Neuroprotocols 1:133-139). Reaction mixtures contained 50 mM Hepes, pH 7.5, 100 μM NADPH, 0.003 mg/mL calmodulin, 1 mM EGTA, 2 mM Ca²⁺, 100 μM L-arginine, and 5 μM oxyhemoglobin, and 0.15 μM BH₄ and were initiated by the addition of nNOS sample. Methemoglobin formation was monitored against a reference cuvette containing complete reaction mixture without enzyme. NO production was calculated using an extinction coefficient of 38 mM⁻¹ cm⁻¹ (Noack et al.,1992).

Citrulline Formation Assay.

Citrulline formation by NOS isoforms was measured in 150 μL reaction mixtures containing 30 mM Hepes, pH 7.5, 1 mM DTT, 120 nM L-[2,3-³H]-arginine without or with unlabeled arginine, 1 mM EGTA, 0.85 mM Ca²⁺ (when present), 6 μM CaM (calmodulin, when present), 100 μM NADPH, and 300 μM BH₄. Incubations were initiated by the addition of enzyme and reaction mixtures incubated for 30 min at 30° C. in duplicate and reactions stopped and analyzed as described by Wolff and Datto (Wolff and Datto, 1992). When citrulline formation was measured as a function of CaM concentration 1.5 mM Ca²⁺ was employed in the assay Assay of NADPH-Oxidase Activity.

Standard reaction mixtures of 1 mL volume in quartz cuvettes contained 50 mM Hepes, pH 7.4, 150 μM NADPH, 6 μM CaM, 1 mM EGTA, 0.85 mM Ca²⁺ and 0.15 μM BH₄. Reactions were initiated by the addition of nNOS enzyme and NADPH consumption was measured from the change in light absorbance at 340 nm as compared to an identical reference cuvette without enzyme using an extinction coefficient of 6.22 mM⁻¹ cm⁻¹.

Cytochrome c Reductase Assay.

The cytochrome c reductase activity of nNOS was determined as described previously, (Cooper et al. (2000) Arch. Biochem. Biophys. 375:183-194) in the presence of 1 mM EGTA, 2 mM Ca²⁺, and 10 μg/ml CaM. An extinction coefficient of 21 mM⁻¹ cm⁻¹ (22) was used to quantify cytochrome c reduction.

Determination of Hydrogen Peroxide.

Hydrogen peroxide was determined by the formation of ferric thiocyanate as described by Heinzel et al. (1992) Biochem. J. 281:627-630. NO synthase was incubated at 30° C. for 20 min in 500 mL of 50 mM Hepes, pH 7.4, 0.5 mM DTT, 200 μM NADPH, 1 mM EGTA, 2 mM Ca²⁺, and 2 μM CaM. Reactions were terminated by the addition of 300 μL of HCl (12 M). The color was developed by addition of 60 μL of ferrous ammonium sulfate (50 mM) and 90 μL of sodium thiocyanante (2M). Samples were incubated for 10 min. at ambient temperature followed by the immediate determination of light absorbance at 492 nm, Blank values were determined in the presence of enzyme but with 20 μg of catalase present. These values were identical to values stopped at zero time. Values were determined by comparison to a standard curve containing 0.25-25 μM hydrogen peroxide.

Preparation and Assay of CaM-Dependent Calcineurin Phosphatase.

Calcineurin phosphatase was prepared from bovine brain extract by the procedures of Wolff, D. J. and Sved, D. W. (1985) J. Biol. Chem. 260:4195-4202. Standard incubations for assay of phosphatase activity were conducted in 1-mL polystyrene disposable cuvettes containing 20 mM p-nitrophenyl phosphate, 50 mM Hepes, pH 7.5, 200 μM MnCl₂, 0.5 mM DTT, and CaM as indicated. Reactions were initiated by addition of enzyme and the formation of p-nitrophenol measured as in the increase in light absorbance at 405 nm.

Preparation of Dansylated Calmodulin.

Dansylated CaM was prepared essentially by the procedure of Johnson, J. D. and Wittenauer, L. A.(1983) Biochem. J. 211:473-479. CaM (10 mg) was dissolved in 2 mL of 10 mM Hepes, pH 7.5, 100 μM Ca²⁺ and was incubated with a 5-fold molar excess of dansyl chloride dissolved in 95% ethanol. The solution was incubated at 25° C. for 6 h and at 4° C. overnight. The dansylated CaM was resolved from free reagent by dialysis versus two changes of a 2000-fold volume excess of deionized water.

Measurement of Nitric Oxide Formation by GH3Pituitary Cells.

Adherent, confluent GH3 cellular monolayers were incubated at 37° C. in 2 mL of Ham's F-10 medium containing 100 μM arginine, 5 μM oxyhemoglobin, 1 μM Bay k 8644, 25 mM KCl, and 1 mM Ca²⁺. Incubations were conducted for 30 min. NO formation was assessed by measuring the formation of methemoglobin as the absorbance difference at 401 nM (absorbance maximum) and 411 nm (isobestic point) as described by Cooper et al. (1998) Arch. Biochem. Biophys. 357:195-206. The nanomoles of NO formed was calculated using an extinction coefficient of 38 mM⁻¹ cm⁻¹.

Sucrose Density Gradient Centrifugation.

Samples of nNOS were either unadjusted (control) or were adjusted to 50 μM C₃-tris-malonyl-C₆₀-fullerene and were applied to a 5-20% sucrose gradient containing 50 mM Mops, pH 7.4, 150 mM NaCl, 1 mM EGTA, 1 mM DTT, 0.3% CHAPSO, and 10 μM BH₄, either without (control) or containing 50 μM C₃-tris-malonyl-C₆₀-fullerene. Gradients were run at 35,000 rpm for 18 h at 4° C. in an L5-L5 Beckman Ultracentrifuge using an SW41 rotor. Fractions (375 μL) were collected using an ISCO density gradient fractionator, Model 185. The fractions from the gradient were kept on ice and portions were assayed for cytochrome c reductase activity (measures both monomers and dimers) and citrulline forming activity (measures dimers exclusively). Hemoglobin (4.09S), catalyze (11.9S), and thyoglobulin (19.4S) were run in a separate tube as sedimentation markers.

Miscellaneous Procedures.

Protein concentrations were measured by the well-known Bradford procedure using bovine serum albumin as the standard. Native gel electrophoresis was performed on 20×20-cm slabs in 20% acrylamide running gels essentially by the procedure and under the conditions described by Davis, B. J. et al.(1964) Ann. N.Y. Acad. Sci. 121:404-427. SDS-PAGE was performed on 20×20-cm slabs in 20% acrylamide running gels essentially by the procedure and under the conditions described by Laemmli, U. K. (1970) Nature (London) 227:680-685.

Those of ordinary skill in the art will appreciate that materials, methods, techniques and procedures other than those specifically described herein can be employed in the practice of this invention without resort to undue experimentation. Those of ordinary skill in the art are aware of various functional equivalents of the methods, techniques and procedures specifically described herein and will appreciate that such equivalents can be employed in the practice of this invention. All such art-recognized functional equivalents are intended to be encompassed in this invention. All references cited herein are incorporated by reference herein in their entirety.

We claim:

1. A method for inhibition of a nitric oxide synthase which comprises the step of contacting the nitric oxide synthase with a water-soluble fullerene derivative which carries one or more water-solublizing substituents in an amount that is effective for inhibiting the nitric oxide synthase.

2. The method of claim 1 wherein the water-soluble fullerene derivative carries a defined number of substituents.

3. The method of claim 1 wherein the water-soluble fullerene derivative carries one water-solublizing substituent.

4. The method of claim 1 wherein the water-soluble fullerene derivative carries three water solubilizing substituents.

5. The method of claim 1 wherein the water-soluble fullerene derivative carries six water-solubilizing substituents.

6. The method of claim 1 wherein the water-soluble fullerene derivative carries at least one substituent that is positively charged, negatively charge or zwitterionic.

7. The method of claim 1 wherein the water soluble fullerene derivative carries at least one substituent that carries an amine group.

8. The method of claim 1 wherein the water-soluble fullerene derivative carries one, three or six substituents each of which carries at least one amine group.

9. The method of claim 1 wherein the water-soluble fullerene derivative carries at least one substituent which is a polyol having two or more OH groups.

10. The method of claim 1 wherein the water-soluble fullerene derivative carries one, three or six substituents each of which is a polyol having two or more OH groups.

11. The method of claim 1 wherein the water-soluble fullerene derivative carries at least one substituent which comprises an ester group.

12. The method of claim 1 wherein the water-soluble fullerene derivative carries one, three or six substituents each of which comprises an ester group.

13. The method of claim 1 wherein the water-soluble fullerene derivative has the schematic formula: 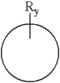
where the circle represent the fullerene, y is an integer ranging from 1 to 30 and R, independent of other R attached to the fullerene, can be selected from the group consisting of —H, —OH, —SH, —(CH2—O)nX, —O—(CH2—O)nX, —(CH2)n—C(R1)(R2)(R3), —O—(CH2)n—C(R1)(R2)(R3), —C(R1)(R2)(R3), —O C(R1)(R2)(R3), -halide —(CH2)n—N(R4)2, —O—(CH2)n—N(R4)2, —NH—(CH2)n—N(R4)2, —(CH2)n—N(R4)3+, —O—(CH2)n—N(R4)3+, —NH—(CH2)n—N(R4)3+, —(CH2)n—OH, —O—(CH2)n—OH, —NH—(CH2)n—OH, —C(O)OR1, —C(O)N(R1)(R2), alkyl groups, alkoxy groups, thioakyl groups (—SR) or aryl groups optionally substituted with one or more of —NH2, —NO2, —OH, —CH2—OH, —(CH2—O)nX1, -halide, akylhalide, —N(R4)2, —N(R4)3+, —COOH, —COO⁻, —SO3−, —SO3H, —PO(OR4)2, —PO2OR⁻, or —PO₃²⁻, one or more bridging substituents:
—N(R1)(R2)—(CH2)n—N(R3)—;
—C(R1)(R2)—N(R5)(R6)+—C(R1)(R2)—;
>C(COOR2)2,
>C(R1)(COOR2);
>C(R1)(CON(R2)(R3));
>C(CON(R2)(R3))2;
>C(P(O)(OR4)2)2, or
>C(P(O)(OR4)2)(P(O)(OR4)O—;
one or more amino acids or peptides;
one or more protected esters or protected amines,
one or more polyethylene glycols,
one or more polyols,
one or more water-solubilizing polymers carrying a plurality of polar, charged or zwitterionic groups, and salts thereof
where n is an integer ranging from 1 to about 200, where R₁₋₃, independently, are selected from the group consisting of —H, —OH, —NH₂, -optionally substituted alkyl, optionally substituted aryl, —SO₃H, —SO₃⁻, —P(O)(OH)₂, —P(O)₂(OH)⁻—COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl, optionally substituted aryl, —CH₂—OH, —COOH, —COO⁻, —CH₂—N(R₄)₂; X₁ is selected from the groups consisting of, —H, -alkyl, —CH₂—OH, the symbol “>” indicates two bonds to the fullerene, R₅ and R₆, independently, can be selected from the same groups as R₄; and one or more of R₁₋₆ can be an amino acid or a peptide optionally bonded to the fullerene in such a way that the amino acid or peptide retains its zwitterionic character with the proviso that at least one of R is a water-solubilizing substituent.

14. The method of claim 13 wherein y is an integer from 1-6.

15. The method of claim 13 wherein y is 1, 3 or 6.

16. The method of claim 13 wherein R is one or more of the bridging substituents:
—N(R₁)(R₂)—(CH₂)ₙ—N(R₃)—;
—C(R₁)(R₂)—N(R₅)(R₆)⁺—C(R1)(R₂)—;
>C(COOR₂)₂,
>C(R₁)(COOR₂);
>C(R₁)(CON(R₂)(R₃));
>C(CON(R₂)(R₃))₂;
>C(P(O)(OR₄)₂)₂, or
>C(P(O)(OR₄)₂)(P(O)(OR₄)O⁻.

17. The method of claim 16 wherein R₁₋₆ are selected from —H or alkyl groups.

18. The method of claim 16 wherein R is one or more of the bridging substituents:
>C(COOR₂)₂,
>C(R₁)(COOR₂);
>C(R₁)(CON(R₂)(R₃)); or
>C(CON(R₂)(R₃))₂.

19. The method of claim 18 wherein R₁₋₆ are selected from —H or alkyl groups.

20. The method of claim 13 wherein all R substituents are water-solubilizing substituents.

21. The method of claim 20 wherein y is 1, 3 or 6.

22. The method of claim 20 wherein R substituents comprise one or more —N(R₄)₂ or —N(R₄)₃₊ groups.

23. The method of claim 20 wherein R substituents comprise one or more zwitterionic groups.

24. The method of claim 20 wherein R substituents comprise one or more —COO— groups, one or more ester or one or more amide groups.

25. The method of claim 1 wherein the 1 wherein the water-soluble fullerene derivative has the schematic formula: 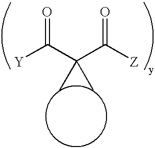
where the circle represent the fullerene, y is an integer ranging from 1 to 30 and Y and Z, independent of other Y and Z attached to the fullerene, can be selected from the group consisting of —H, —OH, —SH, —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(R₃), —O C(R₁)(R₂)(R₃), -halide, —(CH₂)ₙ—N(R₄)₂, —O—(CH₂)ₙ—N(R₄)₂, —NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺, —(CH₂)ₙ—OH, —O—(CH₂)ₙ—OH, —NH—(CH₂)ₙ—OH, —C(O)OR_{1,} —C(O)N(R₁)(R₂), alkyl groups, alkoxy groups, thioakyl groups (—SR₁) or aryl groups optionally substituted with one or more of —NH₂, —NO₂, —OH, —CH₂—OH, —(CH₂—O)ₙX₁, -halide, akylhalide, —N(R₄)₂, —N(R₄)₃⁺, —COOH, —COO⁻, —SO₃^{—}, —SO₃H, —PO(OR₄)₂, —PO₂OR⁻, or —PO₃²⁻,
one or more amino acids or peptides;
one or more protected esters or protected amines,
one or more polyethylene glycols,
one or more polyols,
one or more water-solubilizing polymers carrying a plurality of polar, charged or zwitterionic groups, and salts thereof
where n is an integer ranging from 1 to about 200, where R₁₋₃, independently, are selected from the group consisting of —H, —OH, —NH₂, -optionally substituted alkyl, optionally substituted aryl, —SO₃H, —SO₃⁻, —P(O)(OH)₂, —P(O)₂(OH)⁻—COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl, optionally substituted aryl, —CH₂—OH, —COOH, —COO⁻, —CH₂—N(R₄)₂; X₁ is selected from the groups consisting of, —H, -alkyl, —CH₂—OH, and one or more of R₁₋₄ can be an amino acid or a peptide optionally bonded in the substituent in such a way that the amino acid or peptide retains its zwitterionic character with the proviso that at least one of Y or Z contains a water-solublizing group.

26. The method of claim 25 wherein y is an integer from 1-6.

27. The method of claim 25 wherein y is 1, 3 or 6.

28. The method of claim 25 wherein Y and Z are selected from the group consisting of: —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(R₃), —O C(R₁)(R₂)(R₃), —(CH₂)ₙ—N(R₄)₂, —O—(CH₂)ₙ—N(R₄)₂, —NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺¹, —(CH₂)ₙ—OH, —O—(CH₂)ₙ—OH, —NH—(CH₂)ₙ—OH, —C(O)OR₁, —C(O)N(R₁)(R₂), and optionally substituted alkyl groups, alkoxy groups, thioakyl groups (—SR₁), aryl groups or aryloxy groups (—O-aryl) wherein optional substituents are one or more of —NH₂, —NO₂, —OH, —CH₂—OH, —(CH₂—O)ₙX₁, -halide, -akylhalide, —N(R₄)₂, —N(R₄)₃⁺, —COOH, —COO⁻, —SO₃⁻, —SO₃H, —PO(OR₄)₂, —PO₂OR⁻, or —PO₃²⁻.

29. The method of claim 25 wherein Y and Z are selected from the group consisting of —O—(CH₂—O)ₙX, —O—(CH₂)ₙ—C(R₁)(R₂)(R₃), —O C(R₁)(R₂)(R₃), —O—(CH₂)ₙ—N(R₄)₂, —NH—(CH₂)ₙ—N(R₄)₂, —O—(CH₂)ₙ—N(R₄)₃⁺, —NH—(CH₂)ₙ—N(R₄)₃⁺, —O—(CH₂)ₙ—OH, —NH—(CH₂)ₙ—OH, and optionally substituted alkyl groups, alkoxy groups aryl groups or aryloxy groups (—O-aryl) wherein optional substituents are one or more of —NH₂, —NO₂, —OH, —CH₂—OH, —(CH₂—O)ₙX₁, -halide, -akylhalide, —N(R₄)₂, —N(R₄)₃⁺, —COOH, —COO⁻, —SO₃⁻, —SO₃H, —PO(OR₄)₂, —PO₂OR⁻, or —PO₃²⁻.

30. The method of claim 25 wherein Y and Z are selected from the group consisting of —O—(CH₂)ₙ—N(R₄)₂, —NH—(CH₂)ₙ—N(R₄)₂—O—(CH₂)ₙ—N(R₄)₃⁺, —NH—(CH₂)ₙ—N(R₄)₃⁺, —O—(CH₂)ₙ—OH, —NH—(CH₂)ₙ—OH, alkyl groups and alkoxy groups.

31. The method of claim 25 wherein Y and Z are selected from the group consisting of —O—(CH₂)ₙ—N(R₄), —O—(CH₂)ₙ—N(R₄)₃⁺ wherein n is 1-10 and alkoxy groups having from 1-10 carbon atoms.

32. The method of claim 31 wherein y is 1.

33. The method of claim 31 wherein y is 3.

34. The method of claim 31 wherein y is 6.

35. The method of claim 1 wherein the fullerene is C₆₀ or C₇₀.

36. The method of claim 13 wherein the fullerene is C₆₀ or C₇₀.

37. The method of claim 25 wherein the fullerene is C₆₀ or C₇₀.

38. The method of claim 1 wherein the water-soluble fullerene derivative has IC₅₀ for a nitric oxide synthase lower than 500 μM.

39. The method of claim 1 wherein the water-soluble fullerene derivative has IC₅₀ for a nitric oxide synthase lower than 2 μM.

40. The method of claim 1 wherein the water-soluble fullerene derivative has IC₅₀ for a neuronal nitric oxide synthase lower than 2 μM.

41. The method of claim 1 wherein the water-soluble fullerene derivative exhibits selective inhibition of neuronal nitric oxide synthase or inducible nitric oxide synthase.

42. The method of claim 41 wherein the water-soluble fullerene derivative exhibits IC₅₀ for neuronal nitric oxide synthase which is 10-fold or more lower than its IC₅₀ for endothelial nitric oxide synthase.

43. The method of claim wherein the water-soluble fullerene derivative exhibits IC₅₀ for neuronal nitric oxide synthase which is 100-fold or more lower than its IC₅₀ for endothelial nitric oxide synthase.

44. The method of claim 41 wherein the fullerene derivative is a water-soluble mono malonyl adduct of a fullerene.

45. The method of claim 41 wherein the water-soluble fullerene has the formula: 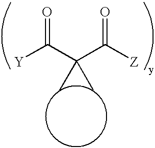
where the circle represent the fullerene, y is 1 and Y and Z, independent of other Y and Z attached to the fullerene, can be selected from the group consisting of —H, —OH, —SH, —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(R₃), —O C(R₁)(R₂)(R₃), -halide, —(CH₂)ₙ—N(R₄)₂, —O—(CH₂)ₙ—N(R₄)₂, —NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺, —(CH₂)ₙ—OH, —O—(CH₂)ₙ—OH, —NH—(CH₂)ₙ—OH, —C(O)OR₁, —C(O)N(R₁)(R₂), alkyl groups, alkoxy groups, thioakyl groups (—SR₁) or aryl groups optionally substituted with one or more of —NH₂, —NO₂, —OH, —CH₂—OH, —(CH₂—O)ₙX₁, -halide, akylhalide, —N(R₄)₂, —N(R₄)₃⁺, —COOH, —COO⁻, —SO₃⁻, —SO₃H, —PO(OR₄)₂, —PO₂OR⁻, or —PO₃²⁻,
one or more amino acids or peptides;
one or more protected esters or protected amines,
one or more polyethylene glycols,
one or more polyols,
one or more water-solubilizing polymers carrying a plurality of polar, charged or zwitterionic groups, and salts thereof
where n is an integer ranging from 1 to about 200, where R₁₋₃, independently, are selected from the group consisting of —H, —OH, —NH₂, -optionally substituted alkyl, optionally substituted aryl, —SO₃H, —SO₃⁻, —P(O)(OH)₂, —P(O)₂(OH)— —COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl, optionally substituted aryl, —CH₂—OH, —COOH, —COO⁻, —CH₂—N(R₄)₂; X₁ is selected from the groups consisting of, —H, -alkyl, —CH₂—OH, and one or more of R₁₋₄ can be an amino acid or a peptide optionally bonded in the substituent in such a way that the amino acid or peptide retains its zwitterionic character with the proviso that at least one of Y or Z contains a water-solublizing group.

46. The method of claim 45 wherein at least one of Y or Z is a charged group or a zwitterionic group.

47. The method of claim 45 wherein both of Y or Z are charged groups or zwitterionic groups.

48. The method of claim 45 wherein one or both of Y or Z are positively charged.

49. The method of claim 48 wherein one or both of Y or Z contain amine cationic groups.

50. The method of claim 45 wherein one or both of Y or Z contain one or more —OH groups.

51. The method of claim 45 wherein one or both of Y or Z are —(CH₂)ₙ—OH, —O—(CH₂)ₙ—OH, —NH—(CH₂)ₙ—OH, or alkyl groups, alkoxy groups, aryl groups or aryloxy groups optionally substituted with one or —OH groups.

52. A pharmaceutical composition for the treatment of a disease or pathological condition that is associated with the levels of nitric oxide in cells or tissues which comprises a pharmaceutically acceptable carrier and one or more water-soluble fullerenes which exhibit an IC₅₀ for a nitric oxide synthase of 500 μM or less present in the composition in an amount or a combined amount effective for inhibition of a nitric oxide synthase.

53. The pharmaceutical composition of claim 52 wherein the water-soluble fullerene derivative exhibits an IC₅₀ for a nitric oxide synthase of 2 μM or less.

54. The pharmaceutical composition of claim 52 wherein the water-soluble fullerene derivative exhibits selective inhibition of neuronal nitric oxide synthase or inducible nitric oxide synthase.

55. The pharmaceutical composition of claim 54 wherein the water-soluble fullerene derivative exhibits IC50 for neuronal nitric oxide synthase which is 10-fold or more lower than its IC50 for endothelial nitric oxide synthase.

56. The pharmaceutical composition of claim 54 wherein the water-soluble fullerene derivative exhibits IC₅₀ for neuronal nitric oxide synthase which is 100-fold or more lower than its IC₅₀ for endothelial nitric oxide synthase.

57. The pharmaceutical composition of claim 54 wherein the water-soluble fullerene is a mono-malonyl adduct.

58. The pharmaceutical composition of claim 54 wherein a substituent of the water-soluble fullerene carries one or more amine groups or cationic amine groups.

59. The pharmaceutical composition of claim 54 wherein a substituent of the water-soluble fullerene carries one or more zwitterionic groups.

60. The pharmaceutical composition of claim 54 wherein a substituent of the water-soluble fullerene carries one or more —OH groups.

61. A method for treating a disease or a pathological condition of an individual that is associated with increased nitric oxide levels in cells or tissues of that individual which comprises the step of administering to that individual a pharmaceutical composition of claim 52.

62. The method of claim 61 wherein the disease or a pathological condition is associated with increased nitric oxide levels in neuronal cells or tissues.

63. The method of claim 62 wherein the water-soluble fullerene derivative of the pharmaceutical composition exhibits selective inhibition of neuronal nitric oxide synthase.

64. The method of claim 63 wherein the water-soluble fullerene derivative exhibits an IC50 value for neuronal nitric oxide synthase that is at least 50-fold lower than its IC50 value for endothelial nitric oxide synthase.

65. The method of claim 63 wherein the water-soluble fullerene derivative exhibits an IC₅₀ value for neuronal nitric oxide synthase that is at least 100-fold lower than its IC50 value for endothelial nitric oxide synthase.

66. The method of claim 63 wherein the water-soluble fullerene derivative is a mono-malonyl adduct.

67. The method of claim 66 wherein the water-soluble fullerene derivative carries one or more charged or zwitterionic groups.

68. The method of claim 66 wherein the water-soluble fullerene derivative carries one or more —OH groups.

67. A method for inhibition of calmodulin which comprises the step of contacting the calmodulin or tissue or cells containing calmodulin with a water-soluble fullerene derivative which carries one or more water-solublizing substituents in an amount that is effective for inhibiting calmodulin.

68. The method of claim 67 wherein the water-soluble fullerene derivative carries 1-6 water-solubilizing substituents.

69. The method of claim 67 wherein the water-soluble fullerene derivative carries at least one substituent that is positively charged, negatively charged or zwitterionic.

70. The method of claim 67 wherein the water soluble fullerene derivative carries at least one substituent that carries an amine group.

71. The method of claim 67 wherein the water-soluble fullerene derivative has the schematic formula: 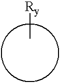
where the circle represent the fullerene, y is an integer ranging from 1 to 30 and R, independent of other R attached to the fullerene, can be selected from the group consisting of —H, —OH, —SH, —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(R₃), —O C(R₁)(R₂)(R₃), -halide —(CH₂)ₙ—N(R₄)₂, ——(CH₂)ₙ—N(R₄)_{2,} —NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺, —(CH₂)ₙ—OH, —O—(CH₂)ₙ—OH, —NH—(CH₂)ₙ—OH, —C(O)OR₁, —C(O)N(R₁)(R₂), alkyl groups, alkoxy groups, thioakyl groups (—SR) or aryl groups optionally substituted with one or more of —NH₂, —NO₂, —OH, —CH₂—OH, —(CH₂—O)ₙX₁, -halide, akylhalide, —N(R₄)₂, —N(R₄)₃⁺, —COOH, —COO⁻, —SO₃⁻, —SO₃H, —PO(OR₄)₂, —PO₂OR⁻, or PO₃²⁻, one or more bridging substituents:
—N(R₁)(R₂)—(CH₂)ₙ—N(R₃)—;
—C(R₁)(R₂)—N(R₅)(R₆)⁺—C(R₁)(R₂)—;
>C(COOR₂)₂,
>C(R₁)(COOR₂);
>C(R₁)(CON(R₂)(R₃));
>C(CON(R₂)(R₃))₂;
>C(P(O)(OR₄)₂)₂, or
>C(P(O)(OR₄)₂)(P(O)(OR₄)O⁻;
one or more amino acids or peptides;
one or more protected esters or protected amines,
one or more polyethylene glycols,
one or more polyols,
one or more water-solubilizing polymers carrying a plurality of polar, charged or zwitterionic groups, and salts thereof
where n is an integer ranging from 1 to about 200, where R₁₋₃, independently, are selected from the group consisting of —H, —OH, —NH₂, -optionally substituted alkyl, optionally substituted aryl, —SO₃H, —SO₃⁻, —P(O)(OH)₂, —P(O)₂(OH)⁻—COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl, optionally substituted aryl, —CH₂—OH, —COOH, —COO⁻, —CH₂—N(R₄)₂; X₁ is selected from the groups consisting of, —H, -alkyl, —CH₂—OH, the symbol “>” indicates two bonds to the fullerene, R₅ and R₆, independently, can be selected from the same groups as R₄; and one or more of R₁₋₆ can be an amino acid or a peptide optionally bonded to the fullerene in such a way that the amino acid or peptide retains its zwitterionic character with the proviso that at least one of R is a water-solubilizing substituent.

72. The method of claim 71 wherein y is an integer from 1-6.

73. The method of claim 72 wherein y is 3.

74. The method of claim 73 wherein a substituent of the water-soluble fullerene carries one or more amine groups or cationic amine groups.

75. The method of claim 73 wherein y is 3 and R is one or more of the bridging substituents:
—N(R₁)(R₂)—(CH₂)ₙ—N(R₃)—;
—C(R₁)(R₂)—N(R₅)(R₆)⁺—C(R₁)(R₂)—;
>C(COOR₂)₂,
>C(R₁)(COOR₂);
>C(R₁)(CON(R₂)(R₃));
>C(CON(R₂)(R₃))₂;
>C(P(O)(OR₄)₂)₂, or
>C(P(O)(OR₄)₂)(P(O)(OR₄)O⁻.

76. The method of claim 75 wherein R₁₋₆ are selected from —H or alkyl groups.

77. The method of claim 73 wherein y is 3 and R is one or more of the bridging substituents:
>C(COOR₂)₂,
>C(R₁)(COOR₂);
>C(R₁)(CON(R₂)(R₃)); or
>C(CON(R₂)(R₃))₂.

78. The method of claim 77 wherein R₁₋₆ are selected from —H or alkyl groups.

79. The method of claim 71 wherein y is 3 and R substituents comprise one or more zwitterionic groups.

80. The method of claim 1 wherein the 1 wherein the water-soluble fullerene derivative has the schematic formula: 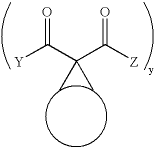
where the circle represent the fullerene, y is an integer ranging from 1 to 30 and Y and Z, independent of other Y and Z attached to the fullerene, can be selected from the group consisting of —H, —OH, —SH, —(CH₂—O)ₙX, —O—(CH₂—O)ₙX, —(CH₂)ₙ—C(R₁)(R₂)(R₃), —O—(CH₂)ₙ—C(R₁)(R₂)(R₃), —C(R₁)(R₂)(₃), —O C(R₁)(R₂)₃), -halide, —(CH₂)ₙ—N(₄)₂, —O—(CH₂)ₙ—N(R₄)₂—NH—(CH₂)ₙ—N(R₄)₂, —(CH₂)ₙ—N(R₄)₃⁺, —O—(CH₂)ₙ—N(R₄)₃⁺, —NH—(CH₂)ₙ—N(R₄)₃⁺, —(CH₂)ₙ—OH, —O—(CH₂)ₙ—OH, —NH—(CH₂)ₙ—OH—C(O)OR₁, —C(O)N(R₁)(R₂), alkyl groups, alkoxy groups, thioakyl groups (—SR₁) or aryl groups optionally substituted with one or more of —NH₂, —NO₂, —OH, —CH₂—OH, —(CH₂—O)ₙX₁, -halide, akylhalide, —N(R₄)₂, N(R₄)₃⁺, —COOH, —COO⁻, —SO₃⁻, —SO₃H, —PO(OR₄)₂, —PO₂OR⁻, or PO₃²⁻,
one or more amino acids or peptides;
one or more protected esters or protected amines,
one or more polyethylene glycols,
one or more polyols,
one or more water-solubilizing polymers carrying a plurality of polar, charged or zwitterionic groups, and salts thereof
where n is an integer ranging from 1 to about 200, where R₁₋₃, independently, are selected from the group consisting of —H, —OH, —NH₂, -optionally substituted alkyl, optionally substituted aryl, —SO₃H, —SO₃⁻, —P(O)(OH)₂, —P(O)₂(OH)⁻—COOH, —COO⁻, —N(R₄)₂, —N(R₄)₃⁺, where R₄ is —H or an optionally substituted alkyl, alkoxy or aryl group; X is selected from the group consisting of —H, -optionally substituted alkyl, optionally substituted aryl, —CH₂—OH, —COOH, —COO⁻, —CH₂—N(R₄)₂; X₁ is selected from the groups consisting of, —H, -alkyl, —CH₂—OH, and one or more of R₁₋₄ can be an amino acid or a peptide optionally bonded in the substituent in such a way that the amino acid or peptide retains its zwitterionic character with the proviso that at least one of Y or Z contains a water-solublizing group.

81. The method of claim 80 wherein y is an integer from 1-6.

82. The method of claim 25 wherein y is 3.

83. A pharmaceutical composition for the treatment of a disease or pathological condition that is associated with modulation of calnodulin activity in cells or tissues which comprises a pharmaceutically acceptable carrier and one or more water-soluble fullerenes which exhibit an IC₅₀ for calmodulin of 250 nM or less present in the composition in an amount or a combined amount effective for inhibition of calmodutin.

84. The pharmaceutical composition of claim 83 wherein the water-soluble fullerene derivative exhibits an IC50 for calmodulin of 100 nM or less.

85. The pharmaceutical composition of claim 83 wherein the water-soluble fullerene is a tris-malonyl adduct.

86. The pharmaceutical composition of claim 85 wherein a substituent of the water-soluble fullerene carries one or more amine groups or cationic amine groups.

87. The pharmaceutical composition of claim 85 wherein a substituent of the water-soluble fullerene carries one or more zwitterionic groups.

88. The pharmaceutical composition of claim 85 wherein a substituent of the water-soluble fullerene carries one or more —OH groups.

89. A method for treating a disease or a pathological condition of an individual that is associated with modulation of calmodulin levels in cells or tissues of that individual which comprises the step of administering to that individual a pharmaceutical composition of claim 83 to inhibit calmodulin activity.

90. The method of claim 89 wherein the water-soluble fullerene derivative is a tris-malonyl adduct.

91. The method of claim 90 wherein the water-soluble fullerene derivative carries one or more charged or zwitterionic groups.

92. The method of claim 90 wherein the water-soluble fullerene derivative carries one or more —OH groups.

93. The method of claim 90 wherein the water-soluble fullerene derivative carries one or more amine groups or cationic amine groups.
